(19) United States
(12) Patent Application Publication
(10) Pub. No.: US 2023/0098432 A1
(43) Pub. Date: Mar. 30, 2023

(54) SYNTHESIS OF GLYCOSYL FLUORIDES

(71) Applicant: CarboCode S.A., Cantanhede (PT)

(72) Inventors: Ferenc Horvath, Pilisszentkereszt (HU); Györgyi Osztrovszky, Kisvarda (HU); Gyula Dekany, Queensland (AU); Agathe Bronikowski, Adelsheim (DE); Piroska Kovacs-Penzes, Jaszbereny (HU); Rafael Soares, Tamengos (PT); Jorge Santos, Alenquer (PT); Fabio Pereira, Cacém (PT); Osama Mahmoud, Konstanz (DE); Nagy Csaba, Konstanz (DE); Dário Jorge Silva Neves, Louriçal (PT)

(21) Appl. No.: 17/801,638

(22) PCT Filed: Feb. 24, 2021
(86) PCT No.: PCT/EP2021/054510

§ 371 Date: Aug. 23, 2022

(30) Foreign Application Priority Data
Feb. 24, 2020 (CH) 00215/20

(51) Int. Cl. C12P 19/18; C07H 5/02; C12N 9/10

(52) U.S. Cl. CPC C12P 19/18; C07H 5/02; C12N 9/1048

(57) ABSTRACT

The present invention relates to a method for producing a glycosyl fluoride of interest, the method comprising providing an internalized exogenous precursor and a genetically modified cell, wherein one or more glycosylation reactions can be performed on the exogenous precursor in the genetically modified cell, the genetically modified cell comprising one or more nucleic acid sequences encoding one or more glycosyltransferase enzymes. The present invention further relates to a compound of the following formula: 2b.

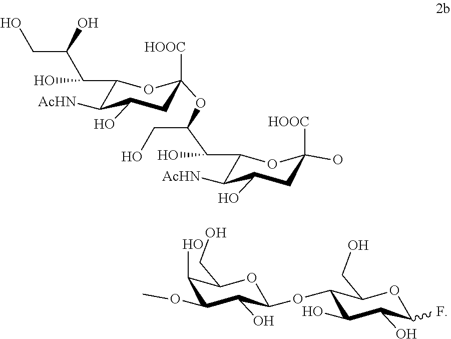

### FIELD OF THE INVENTION

The present invention relates to a method for producing glycosyl fluorides using a biotechnological approach.

### BACKGROUND OF THE INVENTION

Glycosyl fluorides are carbohydrate derivatives which can be described as α-halo ethers. They can be obtained in both anomeric forms, but the α-anomer is the more stable.

Glycosyl fluorides, like other glycosyl halides, are important building blocks for the synthesis of complex oligosaccharides.

Compared to other glycosyl halides, glycosyl fluorides display a remarkable stability. In fact, they are the only glycosyl halides which can be deprotected and dissolved in water. Furthermore, most deprotected glycosyl fluorides are crystalline compounds and can be stored for a long time without decomposition.

Owing to their stability this class of compounds has found many applications both in chemistry and biochemistry (Kazunobu Toshima, Carbohydr. Res. 2000, 327, 15-26; Spencer J. Wiliams, Stephen G. Withers, Carbohydr. Res. 2000, 327, 27-46). For instance, they can be used either as donors or acceptors in chemical glycosylation reactions. Particularly, they can be activated as donors in the aqueous glycosylation of sucrose (Guillaume Pelletier, Aaron Zwicker, C. Liana Allen, Alanna Schepartz, Scott J. Miller, J. Am. Chem. Soc. 2016, 138, 3175-3182) or used in combination with thioglycosides to perform orthogonal glycosylations (Osamu Kanie, Yukishige Ito, Tomoya Ogawa, Tetrahedron Lett. 1996, 37, 4551-4554). Glycosyl-fluorides can also play multiple roles in enzymatic reactions. They have been used as substrate for wild-type and mutant glycosidases, as mechanistic probes and as donors in enzymatic transglycosylation reactions (Spencer J. Wiliams, Stephen G. Withers, Carbohydr. Res. 2000, 327, 27-46).

Chemical synthesis is required for the installation of the fluoride onto the carbohydrate unit. Several methods are available for the fluorination of mono- or di-saccharides (Masataka Yokoyama, Carbohydr. Res. 2000, 327, 5-14). These methods provide easy access to simple glycosyl fluorides which can be used as building blocks for the preparation of complex structures.

Interestingly, due to their stability, glycosyl fluorides can be used as sugar acceptors enabling the preparation of oligosaccharides which retain a good leaving group at their reducing-end and can be used as donors in subsequent glycosylation reactions. Complex oligosaccharides which can function as glycosyl donors are a valuable class of compounds since they can be employed for the preparation of glycoconjugates (US20090170155 A1).

Glycosyl fluoride building blocks can be transformed into complex structures either via chemical synthesis or enzymatic synthesis. However, for the large-scale production of oligosaccharide fluorides, both approaches possess several limitations.

Common challenges connected to chemical synthesis are the control of stereo- and regiochemistry, the need of multiple protecting group manipulations, difficult purification and scale-up.

The enzymatic synthesis of oligosaccharide fluorides has been described (Jamie R. Rich, Anna-Maria Cunningham, Michel Gilbert, Stephen G. Withers Chem. Commun. 2011, 47, 10806-10808). With this method the sugar chain is constructed via sequential glycosylation of a fluoride acceptor catalysed by glycosyltransferases (GTs). Limitations to this approach include engineering the expression and isolating the pure enzyme and the use of expensive glycosyl nucleotide donors.

### SUMMARY OF THE INVENTION

The inventors have established for the first time a biotechnological route for producing complex glycosyl fluorides.
(1) The present invention relates to a method for producing a glycosyl fluoride of interest, the method comprising the steps of:
   a) Providing an exogenous precursor and a genetically modified cell, wherein one or more glycosylation reactions can be performed on the exogenous precursor in the genetically modified cell, the genetically modified cell comprising one or more nucleic acid sequences encoding one or more glycosyltransferase enzymes, and wherein the exogenous precursor is a compound of General Formula I

X—F General Formula I,
wherein
X represents a glycosyl moiety
F represents a fluorine atom
and
X and F are linked by an alpha or a beta glycosidic bond, preferably by an alpha glycosidic bond;
b) Culturing said genetically modified cell in a culture medium comprising said exogenous precursor, whereby
   i. the exogenous precursor is internalized by the cell, and
   ii. one or more glycosylation reactions are performed on the internalized exogenous precursor or on a glycosylated derivative thereof by the one or more glycosyltransferases, to form the glycosyl fluoride of interest,
c) Optionally isolating the glycosyl fluoride of interest from the genetically modified cell and/or from the culture medium.
(2) The method according to (1), wherein the genetically modified cell is a yeast cell or a bacterial cell, preferably an E. coli cell.
(3) The method according to (1) or (2), wherein the one or more glycosyltransferase enzymes comprise one or more sialyltransferases and/or one or more fucosyltransferases.
(4) The method according to (1) or (2), wherein the one or more glycosyltransferase enzymes are selected from the group consisting of β-1,3-N-acetylglucosaminyltransferase, β-1,6-N-acetylglucosaminyltransferase, β-1,3-galactosyltransferase, β-1,4-galactosyltransferase, β-1,4-N-acetylgalactosaminyltransferase, β-1,3-N-acetylgalactosaminyltransferase, β-1,3-glucoronosyltransferase, α-2,3-sialyltransferase, α-2,6-sialyltransferase, α-2,8-sialyltransferase, α-1,2-fucosyltransferase, α-1,3-fucosyltransferase, α-1,4-fucosyltransferase, α-1,4-galactosyltransferase, α-1,3-galactosyltransferase or a combination thereof.
(5) The method according to any one of (1) to (4), wherein the exogenous precursor is a compound of General Formula Ia:

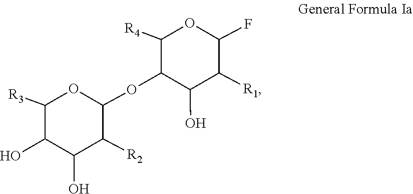

wherein
R₁ and R₂ are independently selected from the group consisting of OH, NH₂ and NH-acyl;
R₃ and R₄ are independently selected from the group consisting of —CH₂—OH and an C₁₋₆ alkyl, preferably methyl.
(6) The method according to any one of (1) to (5), wherein the exogenous precursor is a compound of General Formula Ib:

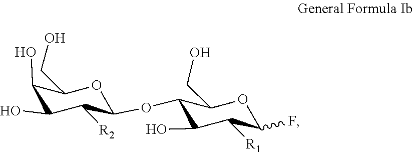

wherein R₁ and R₂ are as defined for General Formula Ia in (5).
(7) The method according to any one of (1) to (6), wherein the exogenous precursor is compound 1a:

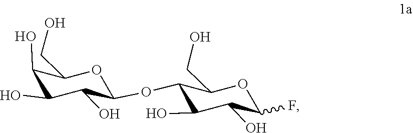

wherein glycosidic bond [symbol] is preferably an alpha glycosidic bond.
(8) The method according to any one of (1) to (7), wherein the genetically modified cell has no β-galactosidase activity.
(9) The method according to any one of (1) to (4), wherein X of General Formula I is a monosaccharide moiety, a disaccharide moiety or a trisaccharide moiety, preferably a monosaccharide moiety or a disaccharide moiety.
(10) The method according to any one of (1) to (5), wherein the glycosyl fluoride of interest is a compound of General Formula IIa:

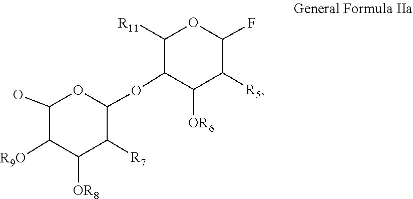

wherein
R₅ and R₇ are independently selected from the group consisting of OH, NH₂, NH-acyl and O-glycoside;
R₆, R₈ and R₉ are independently selected from the group consisting of hydrogen and a glycosyl moiety;
R₁₀ and R₁₁ are independently selected from the group consisting of CH₂—OH, CH₂O-glycoside and C₁₋₆ alkyl, preferably methyl.
(11) The method according to any one of (1) to (6), wherein the glycosyl fluoride of interest is a compound of General Formula III):

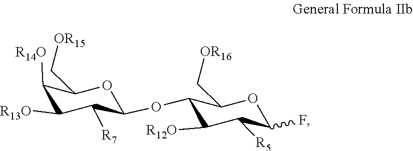

wherein
R₅ and R₇ are independently selected from the group consisting of OH, NH₂, NH-acyl and O-glycoside;
R₁₂, R₁₃, R₁₄, R₁₅, R₁₆, are independently hydrogen or a glycosyl moiety.
(12) The method according to any one of (1) to (7), wherein the glycosyl fluoride of interest is a compound of General Formula IIc:

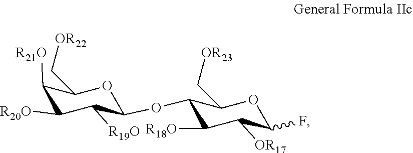

wherein
R₁₇, R₁₈, R₁₉, R₂₀, R₂₁, R₂₂ and R₂₃ are independently hydrogen or a glycosyl moiety.
(13) The method according to (7), wherein the glycosyltransferase enzyme is a α-2,3-sialyltransferase and the produced glycosyl fluoride of interest is compound 2a or a salt thereof:

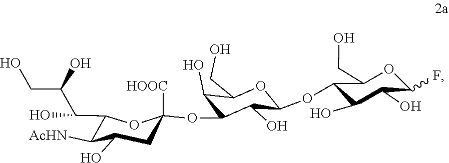

wherein glycosidic bond [symbol] is preferably an alpha glycosidic bond.
(14) The method according to (7), wherein the glycosyltransferase enzymes are α-2,8-sialyltransferase and α-2,3-sialyltransferase, and the produced glycosyl fluoride of interest is compound 2b or a salt thereof:

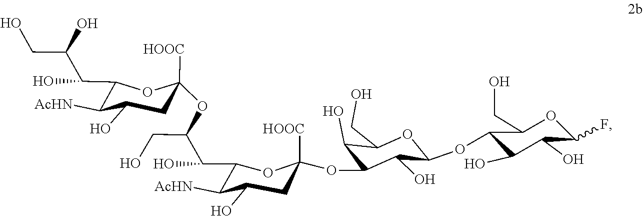

wherein glycosidic bond [symbol] is preferably an alpha glycosidic bond.
(15) The method according to (7), wherein the glycosyltransferase enzymes are β-1,4-N-acetylgalactosaminyltransferase, β-1,3-galactosyltransferase and α-2,3-sialyltransferase, and the produced glycosyl fluoride of interest is compound 2c or a salt thereof:

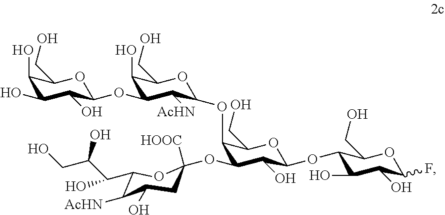

wherein glycosidic bond [symbol] is preferably an alpha glycosidic bond.
(16) A compound 2b or a salt thereof:

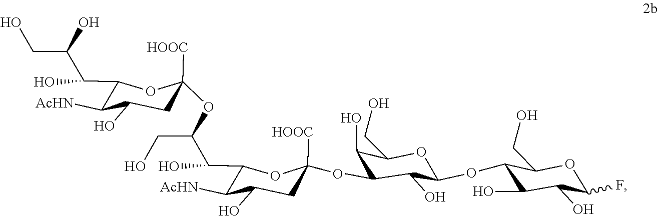

wherein glycosidic bond [symbol] is preferably an alpha glycosidic bond.
(17) A compound selected from:
   Neu5Acα2-3Galβ1-3GalNAcβ1-4Galβ1-4Glc1-F,
   Neu5Acα2-3Galβ1-F,
   Neu5Acα2-3Galβ1-3GalNAcβ1-4(Neu5Acα2-3)Galβ1-4Glc1-F,
   Galβ1-3GalNAcβ1-4(Neu5Acα2-8Neu5Acα2-3)Galβ1-4Glc1-F,
   Neu5Acα2-8Neu5Acα2-3Galβ1-3GalNAcβ1-4Galβ1-4Glc1-F,
   Neu5Acα2-3Galβ1-3(Neu5Acα2-6)GalNAcβ1-4Galβ1-4Glc1-F,
   GalNAcβ1-4(Neu5Acα2-8Neu5Acα2-3)Galβ1-4Glc-F,
   Neu5Acα2-8Neu5Acα2-3Galβ1-4Glc1-F,
   Neu5Acα2-8Neu5Acα2-3Galβ1-3GalNAcβ1-4(Neu5Acα2-3)Galβ1-4Glc1-F,
   Neu5Acα2-3Galβ1-3GalNAcβ1-4(Neu5Acα2-8Neu5Acα2-3)Galβ1-4Glc1-F,
   Galβ1-3GalNAcβ1-4(Neu5Acα2-8Neu5Acα2-8 Neu5Acα2-3)Galβ1-4Glc1-F,
   Neu5Acα2-3Galβ1-3(Neu5Acα2-6)GalNAcβ1-4(Neu5Acα2-3)Galβ1-4Glc1-F,
   GalNAcβ1-4(Neu5Acα2-8Neu5Acα2-8 Neu5Acα2-3)Galβ1-4Glc1-F,
   Neu5Acα2-8Neu5Acα2-8 Neu5Acα2-3Galβ1-4Glc1-F,
   Neu5Acα2-8Neu5Acα2-3Galβ1-3GalNAcβ1-4(Neu5Acα2-8Neu5Acα2-3)Galβ1-4Glc1-F,
   Neu5Acα2-3Galβ1-3 (Neu5Acα2-6)GalNAcβ1-4(Neu5Acα2-8Neu5Acα2-3)Galβ1-4Glc1-F,
   Neu5Acα2-3Galβ1-3GalNAcβ1-4(Neu5Acα2-8Neu5Acα2-8Neu5Acα2-3) Galβ1-4Glc1-F.
(18) A compound selected from:
   Galα1-3Galβ1-4Glc1-F,
   GalNAcβ1-3Galβ1-4Galβ1-4Glc1-F,
   GalNAcβ1-3Galβ1-3Galβ1-4Glc1-F,
   Galβ1-3GalNAcβ1-3Galα1-4Galβ1-4Glc1-F,
   Neu5Acα2-3Galβ1-3GalNAcβ1-3Galβ1-4Galβ1-4Glc1-F,
   Neu5Acα2-3Galβ1-3 (Neu5Acα2-6)GalNAcβ1-3Galα1-4Galβ1-4Glc1-F,
   GalNAcα1-3GalNAcβ1-3Galβ1-4Galβ1-4Glc1-F,
   GalNAcβ1-3GalNAcβ1-3Galβ1-4Galβ1-4Glc1-F,
   GalNAcα1-3GalNAcβ1-3 (Galβ1-3GalNAcβ1-4)Galα1-4Galβ1-4Glc1-F,
   Galβ1-3GalNAcβ1-3Galα1-4Galβ1-4Glc1-F,
   Fucα1-2Galβ1-3GalNAcβ1-3Galβ1-4Galβ1-4Glc1-F,
   GalNAcα1-3(Fucα1-2)Galβ1-3GalNAcβ1-3Galβ1-4Galβ1-4Glc1-F,
   Galα1-3(Fucα1-2)Galβ1-3GalNAcβ1-3Galβ1-4Galβ1-4Glc1-F,
   Galβ1-4(Fucα1-3)GlcNAcβ1-6(Galβ1-3)GalNAcβ1-3Galβ1-4Galβ1-4Glcβ1-F.
(19) A compound selected from:
   GlcNAcβ1-3Galβ1-4GlcNAcβ1-3Galβ1-4Glc1-F,
   Galβ1-4GlcNAcβ1-3Galβ1-4GlcNAcβ1-3Galβ1-4Glc1-F,
   Galβ1-4(Fucα1-3)GlcNAcβ1-3Galβ1-4Glc1-F,
   Galβ1-4(Fucα1-3)GlcNAcβ1-3Galβ1-4GlcNAcβ1-3Galβ1-4Glc1-F,
   Galβ1-4(Fucα1-3)GlcNAcβ1-3Galβ1-4(Fucα1-3)GlcNAcβ1-3Galβ1-4Glc1-F,
   Galβ1-4GlcNAcβ1-3 (Galβ1-4GlcNAcβ1-6)Galβ1-4GlcNAcβ1-3Galβ1-4Glc1-F,
   Fucα1-2Galβ1-4GlcNAcβ1-3Galβ1-4Glc1-F,
   GalNAcβ1-3(Fucα1-2)Galβ1-4GlcNAcβ1-3Galβ1-4Glc1-F,
   GalNAcα1-3(Fucα1-2)Galβ1-4GlcNAcβ1-3Galβ1-4Glc1-F,
   Galα1-3(Fucα1-2)Galβ1-4GlcNAcβ1-3Galβ1-4Glc1-F,
   Fucα1-2Galβ1-3GalNAcα1-3(Fucα1-2)Galβ1-4GlcNAcβ1-3Galβ1-4Glc1-F,
   Fucα1-2Galβ1-4(Fucα1-3)GlcNAcβ1-3Galβ1-4Glc1-F,
   Fucα1-2Galβ1-4(Fucα1-3)GlcNAcβ1-3Galβ1-4GlcNAcβ1-3Galβ1-4Glc1-F,
   Galβ1-3(Fucα1-4)GlcNAcβ1-3Galβ1-4(Fucα1-3)GlcNAcβ1-3Galβ1-4Glc1-F,
   GalNAcβ1-3Galβ1-4GlcNAcβ1-3Galβ1-4Glc1-F,
   Galα1-3Galβ1-4GlcNAcβ1-3 (GalNAcβ1-4)Galβ1-4Glc1-F,
   GlcNAcβ1-3Galβ1-4(Fucα1-3)GlcNAcβ1-3Galβ1-4Glc1-F,
   Galα1-3Galβ1-4(Fucα1-3)GlcNAcβ1-3Galβ1-4Glc1-F,
   Galβ1-4Galβ1-4GlcNAcβ1-3Galβ1-4Glc1-F,
   GalNAcβ1-3Galβ1-3Galβ1-4GlcNAcβ1-3Galβ1-4Glcβ1-F.
(20) A compound selected from:
   Galβ1-3GlcNAcβ1-3Galβ1-4Glc1-F,
   GlcNAcβ1-3Galβ1-4Glc1-F,
   Galβ1-3 GlcNAcβ1-3 (Galβ1-4GlcNAcβ1-6)Galβ1-4Glc1-F,
   Galβ1-4GlcNAcβ1-3 (Galβ1-4GlucNAcβ1-6)Galβ1-4Glc1-F,
   Fucα1-2Galβ1-4Glc1-F,
   Galβ1-4(Fucα1-3)Glc1-F,
   Fucα1-2Galβ1-4(Fucα1-3)Glc1-F,
   Fucα1-2Galβ1-3GlcNAcβ1-3Galβ1-4Glc1-F,
   Galβ1-3(Fucα1-4)GlcNAcβ1-3Galβ1-4Glc1-F,
   Galβ1-3(Fucα1-3)GlcNAcβ1-3Galβ1-4Glc1-F,
   Galβ1-3GlcNAcβ1-3Galβ1-4(Fucα1-3)Glc1-F,
   Fucα1-2Galβ1-3(Fucα1-4)GlcNacβ1-3Galβ1-4Glc1-F,
   Neu5Acα2-6Galβ1-4Glc-1-F,
   Neu5Acα2-3Galβ1-4(Fucα1,3)Glc1-F,
   Neu5Acα2-3Galβ1-3 GlcNAcβ1-3Galβ1-4Glc1-F,
   Galβ1-3 (Neu5Acα2-6)GlcNAcβ1-3Galβ1-4Glc1-F,
   Neu5Acα2-6Galβ1-3 GlcNAcβ1-3Galβ1-4Glc1-F,
   Neu5Acα2-3Galβ1-3 (Neu5Acα2-6)GlcNAcβ1-3Galβ1-4Glc1-F.
(21) A compound selected from:
   Galβ1-3Galβ1-4Glc-1F,
   Galβ1-6Galβ1-4Glc-1F,
   Galβ1-3GalNAcβ1-4Galβ1-4Glc-1F,
   Fucα1-2Galβ1-3(Fucα1-4)GlcNAcβ1-3Galβ1-4Glc1-F.
(22) A compound of General Formula III:

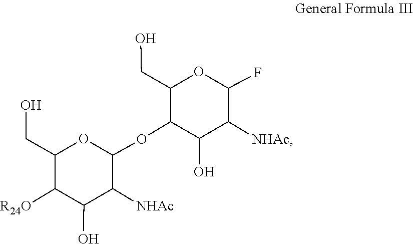

wherein R₂₄ is a glycosyl moiety.
(23) A compound of General Formula IV:

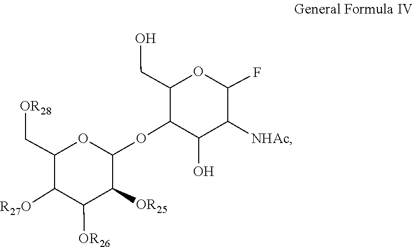

wherein
R₂₅, R₂₆, R₂₇ and R₂₈ are independently H or a glycosyl moiety.
(24) A compound of General Formula V:

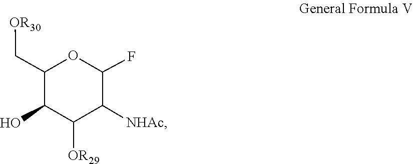

wherein R₂₉ and R₃₀ are either both each a glycosyl moiety, or one of R₂₉ and R₃₀ is a glycosyl moiety and the other one is H.

The present invention overcomes the drawbacks connected to current techniques for the preparation of glycosyl fluorides and provides a new economically attractive method for the synthesis of a broad variety of glycosyl fluorides in cells expressing the required enzymes. This method enables the production of glycosyl fluorides having the desired stereo- and regiochemical configuration without the need for protecting groups manipulations. Purification of the glycosyl fluorides of interest can be achieved without the use of expensive and toxic reagents. Moreover, glycosyltransferases and glycosyl nucleotide donors are directly produced by the engineered cell and thus are readily available. These advantages enable an easy scale-up production of complex glycosyl fluorides.

### DETAILED DESCRIPTION OF THE INVENTION

The present invention relates to a method for producing a glycosyl fluoride of interest, the method comprising the steps of:
a) Providing an exogenous precursor and a genetically modified cell, wherein one or more glycosylation reactions can be performed on the exogenous precursor in the genetically modified cell, the genetically modified cell comprising one or more nucleic acid sequences encoding one or more glycosyltransferase enzymes, and wherein the exogenous precursor is a compound of General Formula I

X—F General Formula I,
wherein
X represents a glycosyl moiety,
F represents a fluorine atom
and
X and F are linked by an alpha or a beta glycosidic bond, preferably by an alpha glycosidic bond;
b) Culturing said genetically modified cell in a culture medium comprising said exogenous precursor, whereby
   iii. the exogenous precursor is internalized by the cell, and
   iv. one or more glycosylation reactions are performed on the internalized exogenous precursor or on a glycosylated derivative thereof by the one or more glycosyltransferases, to form the glycosyl fluoride of interest,
c) Optionally isolating the glycosyl fluoride of interest from the genetically modified cell and/or from the culture medium.

In some embodiments, the genetically modified cell according to the present invention may be prokaryotic or eukaryotic. It may e.g. be a bacterial cell, a yeast cell or a mammalian cell. Preferably, the cell used in the method according to the present invention is a microorganism, such as a bacterium or a yeast. More preferably, the bacterium is selected from the group comprising Escherichia coli, Bacillus spp. (e.g. Bacillus subtilis), Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitidis, Lactobacillus spp., Lactococcus spp., Enterococcus spp., Bifidobacterium spp., Sporolactobacillus spp., Micromomospora spp., Micrococcus spp., Rhodococcus spp. and Pseudomonas, and the yeast is selected from the group comprising Saccharomyces cerevisiae, Schizosaccharomyces pombe, Pichia pastoris and Candida albicans. Most preferably, the genetically modified cell according to the present invention and used in the method according to the present invention is an Escherichia coli (E. coli) cell.

In some embodiments, the genetically modified cell is a yeast cell or a bacterial cell, preferably an E. coli cell.

The genetically modified cell may comprise one or more than one nucleic acid sequence encoding one or more glycosyltransferase enzymes, such as two nucleic acid sequences encoding two or more glycosyltransferase enzymes, or three to five nucleic acid sequences encoding three to five glycosyltransferase enzymes. The nucleic acid sequences may be endogenous or heterologous. When more than one glycosyltransferase enzyme is expressed, the glycosyltransferase enzymes are preferably different ones and accordingly the encoding nucleic acid sequences are preferably different. When more than one glycosyltransferase is expressed and the encoding nucleic acid sequences are different, one or more encoding nucleic acid sequences may be heterologous and one or more encoding nucleic acid sequences may be endogenous.

In a preferred embodiment, the genetically modified cell further comprises one or more nucleic acid sequences encoding one or more epimerase enzymes, wherein the nucleic acid sequences may be endogenous or heterologous.

The origin of the heterologous nucleic acid sequences can be an animal (including humans), a plant, a yeast such as Saccharomyces cerevisiae, Saccharomyces pombe, Candida albicans, a bacterium such as E. coli, Bacillus subtilis, Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitidis, a protozoa such as trypanosoma, or a virus.

In some embodiments, the glycosyltransferase enzymes encoded by the nucleic acid sequence according to the method of the present invention are typically Leloir glycosyltransferases, capable of performing glycosylation reactions between the exogenous precursor or a glycosylated derivative thereof, and an activated sugar nucleotide. The glycosyltransferase enzyme(s) according to the method of the present invention may be a glucosyltransferase, a galactosyltransferase, an N-acetylglucosaminyltransferase, an N-acetylgalactosaminyltransferase, a glucoronosyltransferase, a xylosyltransferase, a mannosyltransferase, a fucosyltransferase, a sialyltransferase, and the like, or combinations thereof.

In some preferred embodiments, the one or more glycosyltransferase enzymes encoded by the one or more nucleic acid sequences according to the method of the present invention comprise one or more sialyltransferases (EC 2.4.99.-). In some more preferred embodiments, the one or more sialyltransferases comprise an α-2,3-sialyltransferase (β-galactoside α-2,3-sialyltransferase (EC 2.44.99.4)), an α-2,6-sialyltransferase (β-galactoside α-2,6-sialyltransferase (EC 2.44.99.1), an α-2,8-sialyltransferase (α-N-acetylneuraminate α-2,8-sialyltransferase (EC 2.44.99.8), or a combination thereof.

In some preferred embodiments, the one or more glycosyltransferase enzymes encoded by the one or more nucleic acid sequences according to the method of the present invention comprise one or more fucosyltransferases. In some more preferred embodiments, the one or more fucosyltransferases comprise an α-1,2-fucosyltransferase (type 1 galactoside α-1,2-fucosyltransferase (EC 2.4.1.69)), an α-1,3-fucosyltransferase (glycoprotein 3- α-L-fucosyltransferase (EC 2.4.1.214), an α-1,4-fucosyltransferase (EC 2.4.1.65), or a combination thereof.

In some preferred embodiments, the one or more glycosyltransferase enzymes encoded by the one or more nucleic acid sequences according to the method of the present invention comprise one or more fucosyltransferases and one or more sialyltransferases.

In some preferred embodiments, the one or more glycosyltransferase enzymes encoded by the one or more nucleic acid sequences according to the method of the present invention may be an, a β-1,3-N-acetylglucosaminyltransferase, a β-1,6-N-acetylglucosaminyltransferase, a β-1,3-galactosyltransferase, a β-1,4-galactosyltransferase, a β-1,4-N-acetylgalactosaminyltransferase, β-1,3-N-acetylgalactosaminyltransferase, a β-1,3-glucoronosyltransferase, an α-2,3-sialyltransferase, an α-2,6-sialyltransferase, an α-2,8-sialyltransferase, an α-1,2-fucosyltransferase, an α-1,3-fucosyltransferase, an α-1,4-fucosyltransferase, an α-1,4-galactosyltransferase, an α-1,3-galactosyltransferase or a combination thereof.

In more preferred embodiments, the one or more glycosyltransferase enzymes encoded by the one or more nucleic acid sequences according to the method of the present invention is a β-1,4-N-acetylgalactosaminyltransferase, a β-1,3-galactosyltransferase, an α-2,3-sialyltransferase, an α-2,8-sialyltransferase or a combination thereof.

In some embodiments, the glycosyltransferase enzyme encoded by the nucleic acid sequence according to the method of the present invention is an α-2,3-sialyltransferase. The nucleic acid sequence according to the method of the present invention encoding the α-2,3-sialyltransferase may be the gene nst from Neisseria meningitidis (GenBank accession number U60660).

In some embodiments, the glycosyltransferase enzymes encoded by the nucleic acid sequences according to the method of the present invention are α-2,8-sialyltransferase and α-2,3-sialyltransferase. The nucleic acid sequence according to the method of the present invention encoding the α-2,8-sialyltransferase and α-2,3-sialyltransferase, respectively, may be the gene cstII from Campylobacter jejuni encoding the bifunctional α-2,3 and α-2,8 sialyltransferase (GenBank accession number AF400048).

In some embodiments, the glycosyltransferase enzymes encoded by the nucleic acid sequences according to the method of the present invention are β-1,4-N-acetylgalactosaminyltransferase, β-1,3-galactosyltransferase and α-2,3-sialyltransferase. The nucleic acid sequence according to the method of the present invention encoding the β-1,4-N-acetylgalactosaminyltransferase, β-1,3-galactosyltransferase and α-2,3-sialyltransferase, respectively, may be the gene cgtA from Campylobacter jejuni (AF130984), the gene from Campylobacter jejuni encoding the β-1,3-galactosyltransferase (AL111168), and gene nst from Neisseria meningitidis (GenBank accession number U60660) respectively.

In some embodiments, the glycosyltransferase enzyme encoded by the nucleic acid sequence according to the method of the present invention is an α-1,2-fucosyltransferase. The nucleic acid sequence according to the method of the present invention encoding the α-1,2-fucosyltransferase may be the gene wbgL from E. coli (UniProt acc. no: E2DNL9), or the gene fucT2 from H. pylori (UniProt acc. no: Q9X3N7).

In some embodiments, the glycosyltransferase enzyme encoded by the nucleic acid sequence according to the method of the present invention is a β-1,3 N-acetyl-glucosyltransferase. The nucleic acid sequence according to the method of the present invention encoding the β-1,3 N-acetyl-glucosyltransferase may be the gene pm1140 (natB) from Pasteurella multocida (UniProt acc. No: F4ZLW7) or the gene lgtA from Neisseria meningitidis (UniProt acc. no: Q8L2V6).

The glycosyl fluorides of interest of the present invention are produced starting from an exogenous precursor. The exogenous precursor is internalized by a cell that expresses one or more glycosyltransferases which catalyze the addition of further monosaccharide units to this exogenous precursor.

In the context of the present application, the following expressions are given a definition that should be taken into consideration with the claims and the description.

The term “substituted” means that the group in question is substituted with a group which typically modifies the general chemical characteristics of the group in question. Preferred substituents include but are not limited to halogen, nitro, amino, azido, oxo, hydroxyl, thiol, carboxy, carboxy ester, carboxamide, alkylamino, alkyldithio, alkylthio, alkoxy, acylamido, acyloxy, or acylthio, each of 1 to 6 carbon atoms, preferably of 1 to 3 carbon atoms. The substituents can be used to modify characteristics of the molecule as a whole such as molecule stability, molecule solubility and an ability of the molecule to form crystals. The person skilled in the art will be aware of other suitable substituents of a similar size and charge characteristics, which could be used as alternatives in a given situation.

In connection with the terms “alkyl” and “acyl”, the term “substituted” means that the group in question is substituted one or several times, preferably 1 to 3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo, C₁₋₆-alkoxycarbonyl, C₁₋₆-alkylcarbonyl, formyl, aryl, aryloxycarbonyl, aryloxy, arylamino, arylcarbonyl, heteroaryl, heteroarylamino, heteroaryloxycarbonyl, heteroaryloxy, heteroarylcarbonyl, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any alkyl, alkoxy, and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

The term “glycosyl moiety” when used herein is defined broadly to encompass a moiety derived from a monosaccharide unit or from an oligosaccharide (more than one monosaccharide units), wherein the anomeric carbon of the monosaccharide or the anomeric carbon at the reducing end of the oligosaccharide is engaged in a glycosidic bond with another chemical entity. A glycosyl moiety having more than one monosaccharide unit may represent a linear or branched structure.

The monosaccharide unit can be any 5-9 carbon atom sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid).

The term “glycosyl moiety” for example includes the following moieties:

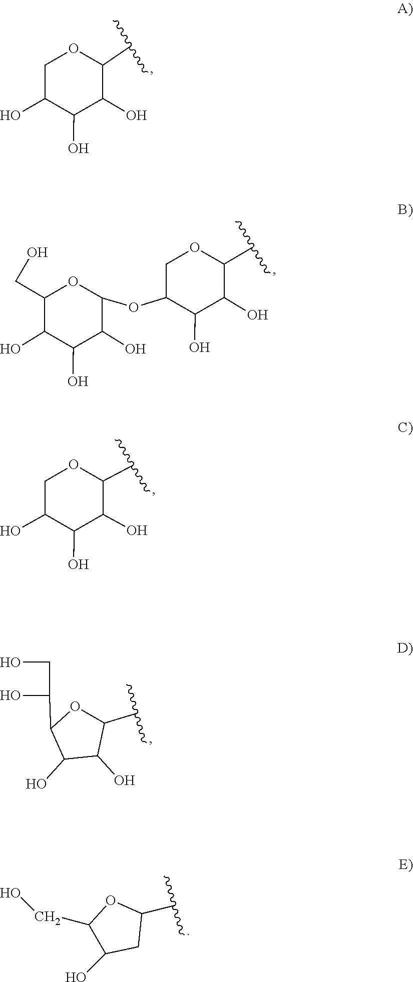 [SMILES: *C1OCC(O)C(O)C1O ; *C1OCC(OC2OC(CO)C(O)C(O)C2O)C(O)C1O ; *C1OCC(O)C(O)C1O ; *C1OC(C(O)CO)C(O)C1O ; *C1CC(O)C(CO)O1]

The term “nucleic acid sequence” refers to a DNA fragment, which is either double-stranded or single stranded, or to a product of transcription of said DNA fragment, and/or to an RNA fragment. A nucleic acid sequence may be naturally present in a cell where it is expressed (termed as “endogenous nucleic acid sequence”) or may be introduced into a cell by recombinant nucleic acid techniques (termed as “heterologous nucleic acid sequence”). Commonly known recombinant nucleic acid techniques are e.g. described in Sambrook et al., Molecular Cloning: A Laboratory Manual, 2′ Ed., Cold Spring Harbor Laboratory Press, Cold Spring Harbor, N.Y. (1989). A heterologous nucleic acid sequence may be a nucleic acid sequence that originates from a source foreign to the particular host cell, or, if from the same source, is modified from its original form. Thus, a heterologous nucleic acid sequence in a cell also includes a nucleic acid sequence that is endogenous to the particular cell but has been subjected to one or more modifications. Modification of a nucleic acid sequence may occur, e.g., by treating the DNA with a restriction enzyme to generate a DNA fragment that is capable of being operably linked to a promoter. Techniques such as site-directed mutagenesis are also useful for modifying a nucleic acid sequence.

The exogenous precursor or exogenous precursor molecule is a glycosyl fluoride represented by General Formula I, preferably by General Formula Ia, more preferably by General Formula Ib, even more preferably compound 1a as outlined in the present invention.

Glycosyl moiety X of General Formula I (and of General Formula Ia and Ib) is in a preferred embodiment a monosaccharide moiety, a disaccharide moiety or a trisaccharide moiety, more preferably a monosaccharide moiety or a disaccharide moiety. Glycosyl moiety X of General Formula I (and of General Formula Ia and Ib) is in a mostly preferred embodiment a disaccharide moiety.

The exogenous precursor molecule is modified by the method of the present invention in the way that one or more further monosaccharide units are attached to it by a glycosidic reaction. The glycosyl fluoride of interest differs from the exogenous precursor in that the glycosyl fluoride of interest comprises at least one more monosaccharide unit as compared to the exogenous precursor.

Glycosyl fluorides are carbohydrates derivatives where a fluoride atom is covalently attached to the anomeric position of the reducing end of the glycosyl moiety.

The fluoride of the exogenous precursor, of the glycosylated derivative of the exogenous precursor and of the glycosyl fluoride of interest may be bound to the glycosyl moiety by either an alpha or a beta glycosidic linkage. An alpha glycosidic linkage is preferred.

The exogenous precursor can be synthesized chemically or enzymatically by any method of producing glycosyl fluorides known to a skilled person. The exogenous precursor is preferably synthesized chemically. The exogenous precursor is preferably lactosyl fluoride, more preferably α-lactosyl fluoride. Example 1 provides an exemplary synthesis of α-lactosyl fluoride.

The genetically modified cell according to the present invention may be prokaryotic or eukaryotic. It may e.g. be a bacterial cell, a yeast cell or a mammalian cell. Preferably, the cell used in the method according to the present invention is a microorganism, such as a bacterium or a yeast. More preferably, the bacterium is selected from the group comprising Escherichia coli, Bacillus spp. (e.g. Bacillus subtilis), Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitidis, Lactobacillus spp., Lactococcus spp., Enterococcus spp., Bifidobacterium spp., Sporolactobacillus spp., Micromomospora spp., Micrococcus spp., Rhodococcus spp. and Pseudomonas, and the yeast is selected from the group comprising Saccharomyces cerevisiae, Schizosaccharomyces pombe, Pichia pastoris and Candida albicans. Most preferably, the genetically modified cell according to the present invention and used in the method according to the present invention is an Escherichia coli (E. coli) cell.

The skilled person will understand that for the method of the present invention, the term “a genetically modified cell” does not intend to mean one single cell, but many cells, typically a cell clone showing the substantially the same genetic characteristics, that are cultured together in a culture medium. In the case of the cell originating from a mammal or from any other multicellular organism, the cells will be cultured in vitro isolated from the organism of origin. The expression “genetically modified” denotes that at least one alteration in the DNA sequence has been performed in the genome of the cell in order to give that cell a specific phenotype. The alteration in the DNA may e.g. be an introduction or a deletion of a DNA fragment in the genome. The alteration in the DNA sequence is herein especially achieved by the expression of a heterologous nucleic acid sequence, in particular a heterologous nucleic acid sequence encoding a glycosyltransferase enzyme. Genome editing may be performed e.g. by commonly known recombinant nucleic acid techniques as e.g. described in Sambrook et al., Molecular Cloning: A Laboratory Manual, 2^{nd} Ed., Cold Spring Harbor Laboratory Press, Cold Spring Harbor, N.Y. (1989). The CRISPR technology may also be used to perform genetic modifications.

The nucleic acid sequence encoding the glycosyltransferase enzyme may be an endogenous nucleic acid sequence or a heterologous nucleic acid sequence, preferably a heterologous nucleic acid sequence.

The genetically modified cell may comprise one or more than one nucleic acid sequence encoding one or more glycosyltransferase enzymes, such as two nucleic acid sequences encoding two or more glycosyltransferase enzymes, or three to five nucleic acid sequences encoding three to five glycosyltransferase enzymes. The nucleic acid sequences may be endogenous or heterologous. When more than one glycosyltransferase enzyme is expressed, the glycosyltransferase enzymes are preferably different ones and accordingly the encoding nucleic acid sequences are preferably different. When more than one glycosyltransferase is expressed and the encoding nucleic acid sequences are different, one or more encoding nucleic acid sequences may be heterologous and one or more encoding nucleic acid sequences may be endogenous.

In a preferred embodiment, the genetically modified cell further comprises one or more nucleic acid sequences encoding one or more epimerase enzymes, wherein the nucleic acid sequences may be endogenous or heterologous.

The origin of the heterologous nucleic acid sequences can be an animal (including humans), a plant, a yeast such as Saccharomyces cerevisiae, Saccharomyces pombe, Candida albicans, a bacterium such as E. coli, Bacillus subtilis, Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitidis, a protozoa such as trypanosoma, or a virus.

The nucleic acid sequences according to the present invention comprise or are a gene, a derivative of a gene or a transcription product of a gene, or a synthetic construct substantially identical to a gene. A derivative of a gene includes a nucleic acid sequence that is a fragment of a gene or a nucleic acid sequence that contains one or more mutations and/or deletions as compared to the original gene, or a cDNA; the mutations or deletions must not strongly impair the function of the encoded enzyme. A derivative of a gene is preferably at least 60% identical to a gene, more preferably at least 90% identical to a gene, even more preferably at least 95% identical to a wildtype gene. The value for gene identity is typically generated when compared and aligned for maximum correspondence, as measured using one of the following sequence comparison algorithms or by visual inspection. A synthetic construct substantially identical to a gene may be produced by synthesis techniques known to the skilled person.

Because of the degeneracy of the genetic code, a large number of functionally identical nucleic acids encode any given peptide or protein. For instance, the codons CGU, CGC, CGA, CGG, AGA and AGG all encode the amino acid arginine. Thus, at every position where an arginine is specified by a codon, the codon can be altered to any of the corresponding codons described without altering the encoded peptide or protein. A derivative of a gene or a synthetic construct substantially identical to a gene is a nucleic acid sequence is in one embodiment codon-optimized for expression in the genetically modified cell according to the present invention

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul el al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands.

In a preferred embodiment, one or more of the nucleic acid sequences according to the present invention are double-stranded DNA fragments. More preferably, the nucleic acid sequences according to the present invention are heterologous nucleic acid sequences. The heterologous nucleic acid sequence is may be placed in an expression cassette. The expression cassette comprises a promoter and the gene or a derivative thereof or synthetic construct to be transcribed. The promoter may be a constitutive or an inducible promoter. A preferred inducible promoter is the lac promoter. The promoter may be induced by addition of the inducer isopropyl β-D-thiogalactoside (IPTG) or by any other lactose analogue to the culture medium. Additional factors or effecting expression may also be used. Transcription start and termination signals, enhancers, and other DNA sequences that influence gene expression can also be included in an expression cassette. When more than one heterologous gene or derivative thereof or synthetic construct is expressed in the cell, the genes or derivatives thereof or synthetic constructs can be expressed on a single expression cassette or on multiple expression cassettes that are compatible and can be maintained in the same cell. When a single expression cassette is used for the expression of more than one heterologous gene or derivative thereof or synthetic construct, the heterologous genes or derivatives thereof or synthetic constructs may be placed under the same promoter, such as an operon, or under several promoters. When several promoters present in one or more expression cassettes are used for the expression of several heterologous genes or derivatives thereof of synthetic constructs, these promoters may be identical or different. Several different inducible promoters present in one or more expression cassettes may be induced by different inducers.

The expression cassette may in one embodiment be introduced into the cell by being placed on an expression vector. The expression vector typically further comprises a selection marker, including e.g. ampicillin or kanamycin.

A heterologous nucleic acid sequence can be expressed in the cell transiently or stably. For example, one expression vector can be used for one or several expression cassettes or more than one expression vector can be used for more than one expression cassette. Heterologous nucleic acid sequences according to the present invention can also be inserted into the chromosome of the cell, using methods known to those skilled in the art, including homologous recombination, site-specific recombination or transposon-mediated gene transposition. The CRISPR technology may also be used to insert one or more heterologous nucleic acid sequences or one or more expression cassettes into a specific locus of the chromosome of the cell. Combinations of expression cassettes in extrachromosomal vectors and expression cassettes inserted into a host cell chromosome can also be used.

Glycosyltransferases are enzymes that catalyze glycosylation reactions between a glycosyl donor, which is typically an activated sugar nucleotide (for Leloir glycosyltransferases), and a glycosyl acceptor, which is a nucleophilic biomolecule including a sugar, a protein or a lipid. Activated sugar nucleotides generally comprise a phosphorylated glycosyl residue attached to a nucleoside. The glycosyl residue of the donor is transferred to the acceptor by a glycosyltransferase, forming a glycosidic linkage.

The glycosyltransferase enzymes encoded by the nucleic acid sequence according to the method of the present invention are typically Leloir glycosyltransferases, capable of performing glycosylation reactions between the exogenous precursor or a glycosylated derivative thereof, and an activated sugar nucleotide. The glycosyltransferase enzyme(s) according to the method of the present invention may be a glucosyltransferase, a galactosyltransferase, an N-acetylglucosaminyltransferase, an N-acetylgalactosaminyltransferase, a glucoronosyltransferase, a xylosyltransferase, a mannosyltransferase, a fucosyltransferase, a sialyltransferase, and the like, or combinations thereof.

In some preferred embodiments, the one or more glycosyltransferase enzymes encoded by the one or more nucleic acid sequences according to the method of the present invention comprise one or more sialyltransferases (EC 2.4.99.-) and/or one or more fucosyltransferases.

In some preferred embodiments, the one or more glycosyltransferase enzymes encoded by the one or more nucleic acid sequences according to the method of the present invention may be an, a β-1,3-N-acetylglucosaminyltransferase, a β-1,6-N-acetylglucosaminyltransferase, a β-1,3-galactosyltransferase, a β-1,4-galactosyltransferase, a β-1,4-N-acetylgalactosaminyltransferase, β-1,3-N-acetylgalactosaminyltransferase, a β-1,3-glucoronosyltransferase, an α-2,3-sialyltransferase, an α-2,6-sialyltransferase, an α-2,8-sialyltransferase, an α-1,2-fucosyltransferase, an α-1,3-fucosyltransferase, an α-1,4-fucosyltransferase, an α-1,4-galactosyltransferase, an α-1,3-galactosyltransferase or a combination thereof.

In more preferred embodiments, the one or more glycosyltransferase enzymes encoded by the one or more nucleic acid sequences according to the method of the present invention is a β-1,4-N-acetylgalactosaminyltransferase, a β-1,3-galactosyltransferase, an α-2,3-sialyltransferase, an α-2,8-sialyltransferase or a combination thereof.

In an especially preferred first embodiment, the glycosyltransferase enzyme encoded by the nucleic acid sequence according to the method of the present invention is an α-2,3-sialyltransferase. The nucleic acid sequence according to the method of the present invention encoding the α-2,3-sialyltransferase may be the gene nst from Neisseria meningitidis (GenBank accession number U60660).

In an especially preferred second embodiment, the glycosyltransferase enzymes encoded by the nucleic acid sequences according to the method of the present invention are α-2,8-sialyltransferase and α-2,3-sialyltransferase. The nucleic acid sequence according to the method of the present invention encoding the α-2,8-sialyltransferase and α-2,3-sialyltransferase, respectively, may be the gene cstII from Campylobacter jejuni encoding the bifunctional α-2,3 and α-2,8 sialyltransferase (GenBank accession number AF400048).

In an especially preferred third embodiment, the glycosyltransferase enzymes encoded by the nucleic acid sequences according to the method of the present invention are β-1,4-N-acetylgalactosaminyltransferase, β-1,3-galactosyltransferase and α-2,3-sialyltransferase. The nucleic acid sequence according to the method of the present invention encoding the β-1,4-N-acetylgalactosaminyltransferase, β-1,3-galactosyltransferase and α-2,3-sialyltransferase, respectively, may be the gene cgtA from Campylobacter jejuni (AF130984), the gene from Campylobacter jejuni encoding the β-1,3-galactosyltransferase (AL111168), and gene nst from Neisseria meningitidis (GenBank accession number U60660) respectively.

The activated sugar nucleotide used for the one or more glycosylation reactions of the present invention performed in the genetically modified cell may e.g. be one or more of UDP-Glc, UDP-Gal, UDP-GlcNAc, UDP-GalNAc, UDP-glucuronic acid, UDP-Xyl, GDP-Man, GDP-Fuc and CMP-sialic acid. The activated sugar nucleotide used for the one or more glycosylation reactions of the present invention is preferably selected from one or more of UDP-Gal, UDP-GalNAc and CMP-sialic acid. A skilled person knows that the choice of glycosyltransferase determines the sugar nucleotide possible as donor for the glycosylation reaction. When more than one different glycosyltransferase enzyme is expressed in the cell, also more than one different activated sugar nucleotide may be needed to be present in the cell, depending on whether the different glycosyltransferases use the same or different sugar nucleotides as donors.

The activated sugar nucleotide is typically synthesized by a suitable nucleotidylyltransferase from a carbon substrate. Accordingly, the genetically modified cell used for the present invention preferably comprises a nucleic acid sequence encoding a nucleotidylyltransferase capable of producing the desired activated sugar nucleotide. The nucleic acid sequence may be naturally present in the cell or may be heterologously expressed after introduction into the cell by means of recombinant techniques generally known to the skilled person. Preferred nucleotidylyltransferases include uridylyltransferases, guanylyltransferases and cytitidylyltransferases. The carbon substrate may be used from an exogenous addition to the genetically modified cell and/or may originate from a salvage pathway. Preferred carbon substrates include glycerol, glucose, glycogen, fructose, maltose, starch, cellulose, pectin, sucrose or chitin.

When a α-2,3-sialyltransferase and/or α-2,8 sialyltransferase are expressed in the genetically modified cell according to the present invention, CMP-sialic acid is typically used as donor for the glycosylation reactions on the exogenous precursor and on a glycosylated derivative thereof, respectively. CMP sialic acid may be produced in the cell from UDP-GlcNAc by the expression of genes encoding a CMP-Neu5Ac synthetase, a sialic acid synthase and a GlcNAc-6-phosphate 2 epimerase while eliminating the activity of N-acetylmannosamine (ManNAc) kinase and N-acetyl-D-neuraminic acid (Neu5Ac) aldolase. The CMP-Neu5Ac synthetase is preferably encoded by the gene neuA from Campylobacter jejuni (AF400048), the sialic acid synthase is preferably encoded by the gene neuB from Campylobacter jejuni (AF400048) and the GlcNAc-6-phosphate 2 epimerase is preferably encoded by the gene neuC from Campylobacter jejuni (AF400048). The genes may be heterologously expressed in the genetically modified cell of the present invention, while, where e.g. E. coli is the genetically modified cell, the nanKETA genes have been inactivated.

When a β-1,4-N-acetylgalactosaminyltransferase, a β-1,3-galactosyltransferase and a α-2,3-sialyltransferase are expressed in the genetically modified cell, UDP-GalNAc is typically used as donor for the glycosylation reaction performed by β-1,4-N-acetylgalactosaminyltransferase, UDP-galactose is typically used as donor for the glycosylation reaction performed by β-1,3-galactosyltransferase, and CMP-sialic acid is typically used as donor for the glycosylation reaction performed by α-2,3-sialyltransferase.

UDP-GalNAc may be produced in the genetically modified cell by the expression of a gene encoding a UDP-GlcNAc-4-epimerase, such as the wbpP gene from Pseudomonas aeruginosa (AF035937) or the gne gene from Campylobacter jejuni (AL111168). CMP sialic acid may be produced in the genetically modified cell from UDP-GlcNAc by the expression of genes encoding a CMP-Neu5Ac synthetase, a sialic acid synthase and a GlcNAc-6-phosphate 2 epimerase while eliminating the activity of N-acetylmannosamine (ManNAc) kinase and N-acetyl-D-neuraminic acid (Neu5Ac) aldolase. The CMP-Neu5Ac synthetase is preferably encoded by the gene neuA from Campylobacter jejuni (AF400048), the sialic acid synthase is preferably encoded by the gene neuB from Campylobacter jejuni (AF400048) and the GlcNAc-6-phosphate 2 epimerase is preferably encoded by the gene neuC from Campylobacter jejuni (AF400048). The genes may be heterologously expressed in the genetically modified cell of the present invention, while, where e.g. E. coli is the genetically modified cell, the nanKETA genes have been inactivated.

Fucosyltransferases, such as α-1,2-fucosyltransferase or α-1,3-fucosyltransferase, typically use GDP-L-fucose as donor for the glycosylation reactions performed in the genetically modified cell according to the present invention. GDP-D-mannose may be converted to GDP-L-fucose by the expression of genes encoding a GDP-mannose 4,6-dehydratase and a GDP-L-fucose synthase. The GDP-mannose 4,6-dehydratase may e.g. be encoded by the gene gmd from E. coli (UniProt acc no: POAC88), the GDP-L-fucose synthase may e.g. be encoded by the gene fcl from E. coli (UniProt acc no: P32055) or by the gene wcaG from E. coli (UniProt acc no: Q8X4R4). GDP-D-mannose may be overproduced e.g. by the recombinant expression of endogenous or heterologous genes encoding a phosphomannomutase and/or a Mannose phosphate guanylyltransferase. The gene encoding the phosphomannomutase may e.g. be manB from E. coli (UniProt acc no: P24175). The gene encoding the Mannose-1-phosphate guanylyltransferase may e.g. be manC from E. coli (UniProt acc no: P24174).

N-acetyl-glucosyltransferases, such as β-1,3 N-acetyl-glucosyltransferase, typically use UDP-GlcNAc as donor for the glycosylation reactions performed in the genetically modified cell according to the present invention. UDP-GlcNAc may be overproduced e.g. by the recombinant expression of endogenous or heterologous genes encoding a phosphoglucosamine mutase, a L-glutamine-D-fructose-6-phosphate aminotransferase, an N-acetylglucosamine-1-phosphateuridyltransferase and/or a glucosamine-1-phosphate acetyltransferase. The gene encoding the phosphoglucosamine mutase may e.g. be glmM from E. coli (UniProt acc no: P31120). The gene encoding the L-glutamine-D-fructose-6-phosphate aminotransferase may e.g. be glmS from E. coli (UniProt acc no: P17169). The gene encoding the N-acetylglucosamine-1-phosphateuridyltransferase and glucosamine-1-phosphate acetyltransferase may be glmU from E. coli (encoding the bifunctional protein GlmU) (UniProt acc no: P0ACC7).

In step b) of the method of the present invention, the genetically modified cell is cultured in a culture medium. When the cell of the present invention is a bacterial or a yeast cell, the culturing corresponds to a fermentation process and the “culture medium” may be also termed as “fermentation broth”.

A fermentation process typically includes two phases:
1. a first phase of exponential cell growth ensured by a carbon-based substrate, and
2. a second phase of cell growth limited by a carbon-based substrate which is added continuously.

The fermentation process may preferably further comprise a third phase (3.) of slowed cell growth obtained by continuously adding to the culture an amount of carbon-based substrate that is less than the amount of carbon-based substrate added in the second phase of the fermentation process so as to further increase the produced compound. More preferably, the amount of carbon-based substrate added during the third phase of the fermentation is at least 30% less than the amount of the carbon-based substrate added during the second phase of the fermentation.

During fermentation, the exogenous precursor may be added to the culture medium at one time point, stepwise or continuously. The pure precursor in solid or in liquid form or a concentrated aqueous solution of the precursor can be added at one time point at the start of fermentation or at the end of the first phase of exponential growth.

The carbon-based substrate may be selected from sucrose, glycerol and glucose. The carbon-based substrate added during the second phase is preferably glycerol.

The culturing is preferably performed under conditions allowing the production of a culture with a high cell density. The skilled person is aware of such conditions, including e.g. pH control and pO₂ control. pO₂ is preferably more than 10%, more preferably more than 20%, even more preferably more than 40% with air flow and stirring.

Further conditions or additions may be applied for the culturing. For example, (NH₄)₂SO₄, MgSO₄, CaCl₂), NH₄HPO₄ or K₂HPO₄ may also be added to the culture, e.g. in a concentration between 2 to 50 g/L, preferably in a concentration between 2 to 25 g/L; they may be added once or several times, e.g. every 12 hours, every 24 hours, every 48 hours, every 72 hours, each time at the same concentration or at different concentrations. One or more of (NH₄)₂SO₄, MgSO₄, CaCl₂), NH₄HPO₄ or K₂HPO₄ may be added to the culture, together or separately. The first phase of the fermentation process may be performed at a reaction temperature of e.g. 30° C., 31° C., 32° C., 33° C., 34° C., 35° C. or 36° C.

The second phase of the fermentation process may be performed at a reaction temperature of e.g. 25° C., 26° C., 27° C., 28° C., 29° C. or 30° C.

The pH regulated may be kept stable by the addition of e.g. aqueous NH₄OH, NaOH or KOH solution.

In step b) i. of the method of the present invention, the exogenous precursor is internalized by the cell. The internalization step must not affect the basic and vital functions or destroy the integrity of the cell. The exogenous precursor molecule may be internalized solely or also via a passive transport during which the exogenous precursor molecule diffuses passively across the plasma membrane of the cell. The flow is directed by the concentration difference in the extra- and intracellular space with respect to the exogenous precursor molecule to be internalized, which exogenous precursor molecule is supposed to pass from the place of higher concentration to the zone of lower concentration tending towards an equilibrium. Typically, the genetically modified cell comprises a transporter protein, which internalizes the exogenous precursor molecule via active transport. Different transporter proteins have specificities for different sugar moieties of the molecules to be internalized. This specificity may be altered by mutation by means of common recombinant DNA techniques. Preferably, the internalization of the exogenous precursor molecule is performed via a transporter protein.

The internalized precursor is then subjected to a glycosylation reaction according to step b) ii) of the method of the present invention. For the glycosylation reaction of the present invention taking place in the cell, the exogenous precursor molecule serves as glycosyl acceptor. The addition of one monosaccharide unit to the exogenous precursor molecule is performed by a glycosyltransferase. The resulting molecule is termed in the present context as “glycosylated derivative of the exogenous precursor” or as “glycosyl fluoride of interest”, depending on whether the molecule is subjected to at least one further glycosylation reaction in the cell (then termed as “glycosylated derivative of the exogenous precursor” or just “glycosylation derivative”) or whether it is the final molecule to be produced and subjected to step c) of the method of the present invention (then termed as “glycosyl fluoride of interest”). If more than one glycosylation reaction is performed in the cell, the “glycosylation derivative” is the acceptor molecule for the second and every further glycosylation reaction. One to five glycosylation reactions are preferably performed in the cell. The monosaccharide units that are added during a second and any further glycosylation reaction may be identical or different. The skilled person will understand that the addition of different monosaccharide units is performed by different glycosyltransferases, which are encoded by different nucleic acid sequences, using different activated sugar nucleotides as donor molecule. The addition of identical monosaccharide units is typically also performed by different glycosyltransferases, which are encoded by different nucleic acid sequences, using however the same activated sugar nucleotides as donor molecule. Accordingly, when the glycosyl fluoride of interest comprises at least two more monosaccharide units as compared to the exogenous precursor, those monosaccharide units are either identical or different from each other.

The glycosyl moiety of the exogenous precursor of the present invention is preferably lactose (Lac, Galβ1-4Glc-) and the exogenous precursor according to the present invention is alpha- or beta-lactosyl fluoride as shown in compound 1a, preferably alpha-lactosyl fluoride.

The glycosyl fluoride of interest of the present invention is preferably selected from the following compounds or from salts thereof:

Galβ1-3GalNAcβ1-4(Neu5Acα2-3)Galβ1-4Glc1-F
Neu5Acα2-3Galβ1-3GalNAcβ1-4Galβ1-4Glc1-F
GalNAcβ1-4(Neu5Acα2-3)Galβ1-4Glc1-F
Neu5Acα2-3Galβ1-4Glc1-F
Neu5Gcα2-3Galβ1-4Glc1-F
Neu5Acα2-3Galβ1-F
Neu5Acα2-3Galβ1-3GalNAcβ1-4(Neu5Acα2-3)Galβ1-4Glc1-F
Galβ1-3GalNAcβ1-4(Neu5Acα2-8Neu5Acα2-3)Galβ1-4Glc1-F
Neu5Acα2-8Neu5Acα2-3Galβ1-3GalNAcβ1-4Galβ1-4Glc1-F
Neu5Acα2-3Galβ1-3(Neu5Acα2-6)GalNAcβ1-4Galβ1-4Glc1-F
GalNAcβ1-4(Neu5Acα2-8Neu5Acα2-3)Galβ1-4Glc1-F
Neu5Acα2-8Neu5Acα2-3Galβ1-4Glc1-F
Neu5Acα2-8Neu5Acα2-3Galβ1-3GalNAcβ1-4(Neu5Acα2-3)Galβ1-
4Glc1-F
Neu5Acα2-3Galβ1-3GalNAcβ1-4(Neu5Acα2-8Neu5Acα2-3)Galβ1-
4Glc1-F
Galα1-4Galβ1-4Glc1-F
Galα1-3Galβ1-4Glc1-F
GalNAcβ1-3Galα1-4Galβ1-4Glc1-F
GalNAcβ1-3Galα1-3Galβ1-4Glc1-F
Galβ1-3GalNAcβ1-3Galα1-4Galβ1-4Glc1-F
Neu5Acα2-3Galβ1-3GalNAcβ1-3Galα1-4Galβ1-4Glc1-F
Neu5Acα2-3Galβ1-3(Neu5Acα2-6)GalNAcβ1-3Galα1-4Galβ1-4Glc1-F
GalNAcα1-3GalNAcβ1-3Galα1-4Galβ1-4Glc1-F
GalNAcβ1-3GalNAcβ1-3Galα1-4Galβ1-4Glc1-F
GalNAcα1-3GalNAcβ1-3(Galβ1-3GalNAcβ1-4)Galα1-4Galβ1-4Glc1-F
Galβ1-3GalNAcβ1-3Galα1-4Galβ1-4Glc1-F
Fucα1-2Galβ1-3GalNAcβ1-3Galα1-4Galβ1-4Glc1-F
GalNAcα1-3(Fucα1-2)Galβ1-3GalNAcβ1-3Galα1-4Galβ1-4Glc1-F
Galα1-3(Fucα1-2)Galβ1-3GalNAcβ1-3Galα1-4Galβ1-4Glc1-F
Galβ1-4(Fucα1-3)GlcNAcβ1-6(Galβ1-3)GalNAcβ1-3Gala1-4Galβ1-
4Glc1-F
Galβ1-4GlcNAcβ1-3Galβ1-4Glc1-F
GlcNAcβ1-3Galβ1-4GlcNAcβ1-3Galβ1-4Glc1-F
Galβ1-4GlcNAcβ1-3Galβ1-4GlcNAcβ1-3Galβ1-4Glc1-F
Galβ1-4(Fucα1-3)GlcNAcβ1-3Galβ1-4Glc1-F
Galβ1-4(Fucα1-3)GlcNAcβ1-3Galβ1-4GlcNAcβ1-3Galβ1-4Glc1-F
Galβ1-4(Fucα1-3)GlcNAcβ1-3Galβ1-4(Fucα1-3)GlcNAcβ1-3Galβ1-
4Glc1-F
Galβ1-4GlcNAcβ1-3(Galβ1-4GlcNAcβ1-6)Galβ1-4GlcNAcβ1-3Galβ1-
4Glc1-F
Fucα1-2Galβ1-4GlcNAcβ1-3Galβ1-4Glc1-F
GalNAcα1-3(Fucα1-2)Galβ1-4GlcNAcβ1-3Galβ1-4Glc1-F
GalNAcβ1-3(Fucα1-2)Galβ1-4GlcNAcβ1-3Galβ1-4Glc1-F
Galα1-3(Fucα1-2)Galβ1-4GlcNAcβ1-3Galβ1-4Glc1-F
Fucα1-2Galβ1-3GalNAcα1-3(Fucα1-2)Galβ1-4GlcNAcβ1-3Galβ1-
4Glc1-F
Fucα1-2Galβ1-4(Fucα1-3)GlcNAcβ1-3Galβ1-4Glc1-F
Fucα1-2Galβ1-4(Fucα1-3)GlcNAcβ1-3Galβ1-4GlcNAcβ1-3Galβ1-
4Glc1-F
Galβ1-3(Fucα1-4)GlcNAcβ1-3Galβ1-4(Fucα1-3)GlcNAcβ1-3Galβ1-
4Glc1-F
Galβ1-3Galβ1-4GlcNAcβ1-3Galβ1-4Glc1-F
GalNAcβ1-3Galβ1-4GlcNAcβ1-3Galβ1-4Glc1-F
Galα1-3Galβ1-4GlcNAcβ1-3(GalNAcβ1-4)Galβ1-4Glc1-F
GlcNAcβ1-3Galβ1-4(Fucα1-3)GlcNAcβ1-3Galβ1-4Glc1-F
Galα1-3Galβ1-4(Fucα1-3)GlcNAcβ1-3Galβ1-4Glc1-F
Galβ1-4Galβ1-4GlcNAcβ1-3Galβ1-4Glc1-F
GalNAcβ1-3Galα1-3Galβ1-4GlcNAcβ1-3Galβ1-4Glc1-F
Galα1-3Galβ1-4GlcNAcβ1-3Galβ1-4Glc1-F
Fucα1-2Galβ1-3(Fucα1-4)GlcNAcβ1-3Galβ1-4Glc1-F
Fucα1-2Galβ1-4Glc1-F
Galβ1-3Galβ1-4Glc1-F
Galβ1-4Galβ1-4Glc1-F
Galβ1-6Galβ1-4Glc1-F
Galβ1-3GalNAcβ1-4Galβ1-4Glc1-F
Galβ1-3GlcNAcβ1-3Galβ1-4Glc1-F
Galβ1-4GlcNAcβ1-3Galβ1-4Glc1-F
Galβ1-3GlcNAcβ1-3(Galβ1-4GlcNAcβ1-6)Galβ1-4Glc1-F
Galβ1-4GlcNAcβ1-3Galβ1-4GlucNAcβ1-3Galβ1-4Glc1-F
Galβ1-4(Fucα1-3)Glc1-F
Fucα1-2Galβ1-4(Fucα1-3)Glc1-F
Fucα1-2Galβ1-3GlcNAcβ1-3Galβ1-4Glc1-F
Galβ1-3(Fucα1-4)GlcNAcβ1-3Galβ1-4Glc1-F
Galβ1-3(Fucα1-3)GlcNAcβ1-3Galβ1-4Glc1-F
Galβ1-3GlcNAcβ1-3Galβ1-4(Fucα1-3)Glc1-F
Neu5Acα2-3Galβ1-4Glc1-F
Neu5Acα2-6Galβ1-4Glc1-F
Neu5Acα2-3Galβ1-4(Fucα1,3)Glc1-F
Neu5Acα2-3Galβ1-3GlcNAcβ1-3Galβ1-4Glc1-F
Galβ1-3(Neu5Acα2-6)GlcNAcβ1-3Galβ1-4Glc1-F
Neu5Acα2-6Galβ1-3GlcNAcβ1-3Galβ1-4Glc1-F
Neu5Acα2-3Galβ1-3(Neu5Acα2-6)GlcNAcβ1-3Galβ1-4Glc1-F
GlcNAcβ1-3Galβ1-4Glc1-F

The above-listed glycosyl fluorides of interest may be alpha- or beta-glycosyl fluorides and may as well be illustrated in the following style:

Galβ1-3GalNAcβ1-4(Neu5Acα2-3)Galβ1-4Glca/β1-F (as exemplified for the compound first listed in the table above).

The glycosyl fluorides listed in the table above are preferably alpha glycosyl fluorides, which may be illustrated as follows:

Galβ1-3GalNAcβ1-4(Neu5Acα2-3)Galβ1-4Glcα1-F (as exemplified for the compound first listed in the table above).

The glycosyl fluoride of interest of the present invention is preferably selected from Neu5Acα2-3Galβ1-4Glc1-F, Neu5Acα2-8Neu5Acα2-3Galβ1-4Glc1-F, Galβ1-3GalNAcβ1-4(Neu5Acα2-3)Galβ1-4Glc1-F, GalNAcβ1-4(Neu5Acα2-3)Galβ1-4Glc1-F, Fucα1-2Galβ1-4Glc1-F and GlcNAcβ1-3Galβ1-4Glc1-F, whose glycosyl moieties correspond to the glycosyl moieties of GM3, GD3, GM1a, GM2, 2′-FL and LNT-II, respectively.

The glycosyl fluoride of interest of the present invention is more preferably selected from Neu5Acα2-3Galβ1-4Glc1-F, Neu5Acα2-8Neu5Acα2-3Galβ1-4Glc1-F and Galβ1-3GalNAcβ1-4(Neu5Acα2-3)Galβ1-4Glc1-F, whose glycosyl moieties correspond to the glycosyl moieties of GM3, GD3 and GM1a, respectively.

The glycosyl fluoride of interest is in some embodiments selected from:

Neu5Acα2-3Galβ1-4Glcα1-F (2d) or a salt thereof:

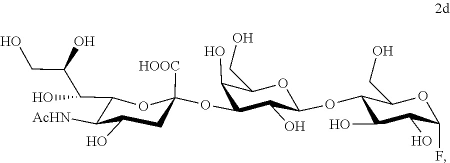

Neu5Acα2-8Neu5Acα2-3Galβ1-4Glcα1-F (2e) or a salt thereof:

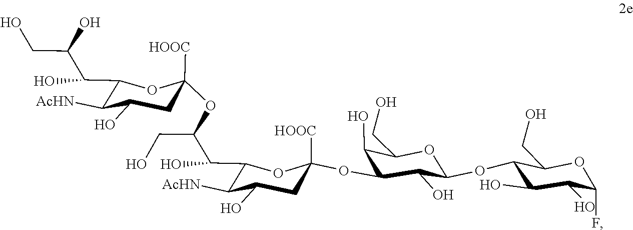

Galβ1-3GalNAcβ1-4(Neu5Acα2-3)Galβ1-4Glcα1-F (2f) or a salt thereof:

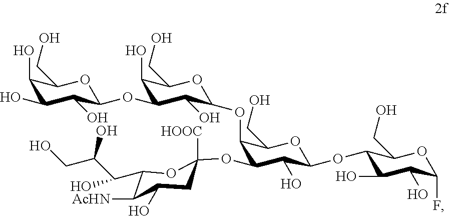

GalNAcβ1-4(Neu5Acα2-3)Galβ1-4Glcα1-F (2g) or a salt thereof:

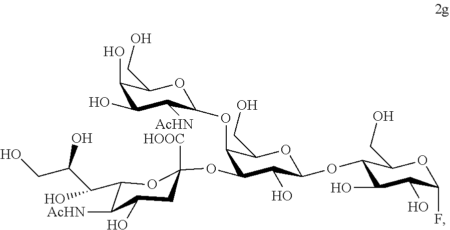

Fucα1-2Galβ1-4Glcα1-F (2h) or a salt thereof:

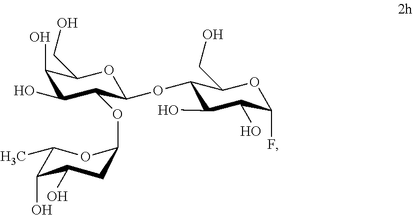

GleNAcβ1-3Galβ1-4Glcα1-F (2i) or a salt thereof:

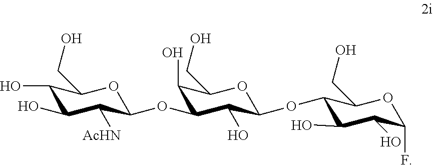

In a preferred embodiment, the genetically modified cell lacks any enzymatic activity which would degrade the exogenous precursor, the glycosylated derivatives of the exogenous precursor and/or the glycosyl fluoride of interest. In a more preferred embodiment, the endogenous gene encoding for β-galactosidase (EC 3.2.1.23), the endogenous gene encoding for N-acetylmannosamine kinase (EC 2.7.1.60) or especially the endogenous genes encoding for β-galactosidase and/or for N-acetylmannosamine is/are inactivated in the genetically modified cell, so as no functional enzyme can be produced. Most preferably, the endogenous gene encoding for β-galactosidase is inactivated in the genetically modified cell. Accordingly, where the genetically modified cell is E. coli, the gene lacZ is preferably inactivated. The gene encoding for α-galactosidase (in E. coli the gene melA) may also be inactivated.

Step c) of the method of the present invention relates to the isolation of the glycosyl fluoride of interest from the cell or from the culture medium. Step c) may be an optional step of the method according to the present invention. The glycosyl fluoride of the method of the present invention can accumulate both in the intra- and extracellular matrix. Glycosyl fluorides having more monosaccharide units tend to accumulate in the cell, while glycosyl fluorides having less monosaccharide units are rather exported from the cell. When exported, the glycosyl fluoride of interest may be exported from the cell via passive transport, by diffusing outside across the cell membrane into the culture medium. The export may be facilitated or mediated by sugar efflux transporters. A sugar efflux transporter may be naturally present in the cell or may be provided in form of a heterologous nucleic acid sequence encoding for the sugar efflux transporter produced by recombinant techniques known to the skilled person. The endogenous or heterologous nucleic acid sequence encoding for the sugar efflux transporter may in one embodiment be mutated by means of known recombinant techniques or may be overexpressed to increase the specificity towards the glycosyl moiety of the glycosyl fluoride of interest to be secreted.

For the isolation step, the culture medium is preferably separated from the cells by filtration or centrifugation. When the glycosyl fluoride of interest is mainly exported from the cell, it is mainly present in the supernatant containing the culture medium and purified and isolated therefrom by means of standard separation, purification and isolation techniques such as crystallization, precipitation and chromatography (e.g. silica, reverse phase, size exclusion, gel and/or ion exchange resin chromatography, etc.). When the glycosyl fluoride of interest accumulates mainly inside the cell, the separated cells are preferably permeabilized. For that, the cells are resuspended in water and subjected to heat and/or acid or base treatment. Sodium hydroxide may be used for a base treatment and sulfuric acid may be used for acid treatment. The glycosyl fluoride of interest is then separated from the treated cells by filtration and purified and isolated from the supernatant by means of standard separation, purification and isolation techniques such as gel and/or ion exchange resin chromatography. The supernatant containing the product from the culture medium may in one embodiment be combined with the supernatant containing the product from the lysed cells. Also in this embodiment, the product may be purified and isolated from the combined supernatant by means of standard separation, purification and isolation techniques such as gel and/or ion exchange resin chromatography.

The invention relates in one preferred embodiment to a method for producing Neu5Acα2-3Galβ1-4Glcα/β1-F (3′-sialyllactosyl fluoride, 2a), the method comprising the steps of:
a) Providing an exogenous precursor and a genetically modified cell, wherein the exogenous precursor is lactosyl fluoride, preferably α-lactosyl fluoride, and wherein the genetically modified cell is an E. coli lacZ⁻ cell comprising a heterologous nucleic acid sequence encoding an α-2,3-sialyltransferase and one or more heterologous nucleic acid sequences encoding a CMP-Neu5Ac synthetase, a sialic acid synthase and a GlcNAc-6-phosphate 2 epimerase, and wherein the nanKETA genes of the E. coli lacZ⁻ cell have been inactivated;
b) Culturing said genetically modified cell in a culture medium comprising said exogenous precursor, whereby
   i. the exogenous precursor is internalized by the cell, and
   ii. one glycosylation reaction is performed on the internalized exogenous precursor, to form 2a,
c) Optionally isolating the 2a from the genetically modified cell and/or the culture medium.

The invention relates in another preferred embodiment to a method for producing Neu5Acα2-8Neu5Acα2-3Galβ1-4Glcα/β1-F (2b)-F, the method comprising the steps of:
a) Providing an exogenous precursor and a genetically modified cell, wherein the exogenous precursor is lactosyl fluoride, preferably α-lactosyl fluoride, and wherein the genetically modified cell is an E. coli lacZ⁻ cell comprising one or more heterologous nucleic acid sequences encoding an α-2,3-sialyltransferase and an α-2,8-sialyltransferase and one or more heterologous nucleic acid sequences encoding a CMP-Neu5Ac synthetase, a sialic acid synthase and a GlcNAc-6-phosphate 2 epimerase, and wherein the nanKETA genes of the E. coli lacZ⁻ cell have been inactivated;
b) Culturing said genetically modified cell in a culture medium comprising said exogenous precursor, whereby
i. the exogenous precursor is internalized by the cell, and
ii. two glycosylation reactions are performed on the internalized exogenous precursor, to form 2b,
c) Optionally isolating the 2b from the genetically modified cell and/or the culture medium.

### EXAMPLES

### Example 1: Preparation of the Genetically Engineered Bacterial Strains Used for Fermentation

### Example 1a: Preparation of the Genetically Engineered Bacterial Strain for the Production of the Glycosyl Fluorides Described in Examples 5 and 6

The engineered E. coli host strain and the transformed plasmids were constructed in accordance with WO 2007/101862 A1, Fierfort et al. Journal of Biotechnology 134, 261-265 (2008) and Priem et al. Glycobiology 12(4), 234-240 (2002); the strain was engineered from an E. coli K12 strain derivative in which the genes lacA and lacZ as well as the genes nanKETA have been deleted and which has been co-transformed with a plasmid carrying the neuABC genes from Campylobacter jejuni, and a second plasmid carrying the α-2,3-sialyltransferase-encoding nst gene from Neisseria meningitidis for the production of α-N-acetylneuroaminosyl-(2→3)-β-D-galactopyranosyl-(1→4)-α-D-glucopyranosyl fluoride (2d) or with a second plasmid carrying the α-2,3 α-2,8-sialyltransferase-encoding cstII gene from Campylobacter jejuni for the production of α-N-acetylneuroaminosyl-(2→8)-α-N-acetylneuroaminosyl-(2→3)-β-D-galactopyranosyl-(1→4)-α-D-glucopyranosyl fluoride (2e), respectively.

### Example 1b: Preparation of the Genetically Engineered Bacterial Strain for the Production of the Glycosyl Fluoride 2h Described in Example 7

The strain was engineered from an E. coli K12 strain derivative in which the genes lacA and lacZ have been deleted and has been transformed with a plasmid carrying the α-1,2-fucosyltransferase-encoding wbgL gene from E. coli.

### Example 1c: Preparation of the Genetically Engineered Bacterial Strain for the Production of the Glycosyl Fluoride 2i Described in Example 8

The strain was engineered from an E. coli K12 strain derivative in which the genes lacA and lacZ as well as the genes nanKETA have been deleted and has been transformed with a plasmid carrying the β-1,3 N-acetyl-glucosyltransferase-encoding natB gene from Pasteurella multocida.

### Example 2: Synthesis of [β-D-galactopyranosyl-(1→4)-α-D-glucopyranosyl fluoride] (1b)

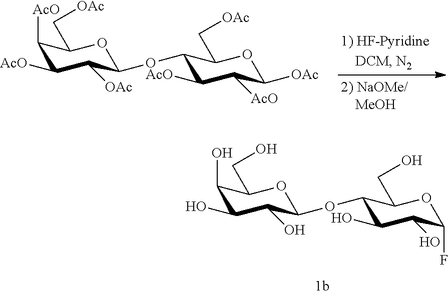

1,2,3,6,2′,3′,4′,6′-octa-O-acetyl-β-D-lactoside (10 g, 14.7 mmol) was dissolved in anhydrous dichloromethane (11 mL) under a nitrogen atmosphere. The flask was cooled to −15° C., and 17 ml of a hydrogen fluoride—pyridine solution was added (HF: 70%; 13.1 g, 0.64 mol). The solution was let to equilibrate to 0° C. during 2 hrs, then the cooling bath was removed, and the mixture was stirred at room temperature for additional 5 hrs. Then, the reaction mixture was poured into a stirred, ice-cold mixture of 50 ml dichloromethane and 100 ml ice-water and stirred until the ice melted. The organic phase was washed with saturated sodium hydrocarbonate (3×200 ml) and brine (2×10 ml), then dried over sodium sulphate and concentrated under vacuum to obtain crude 2,3,6,2′,3′,4′,6′-hepta-O-acetyl-β-D-lactopyranosyl fluoride as a white foam (9.4 g).

The latter was dissolved in 47 ml of dry methanol and a 25% methanolic NaOMe solution was added (168 ml, 0.05 eq). The reaction mixture was stirred at room temperature for 2 hrs. The formed solid was filtered off, washed with isopropanol and dried in a desiccator over phosphorus(V) oxide to afford compound 1b (3.87 g, 76% yield).

NMR in D₂O (ppm)

¹H-NMR: 5.60-5.74 (1H, dd, 53.4 Hz; J₂: 2.8 Hz), 4.44 (1H, d, J: 7.8 Hz), 3.58-3.98 (m, 11H), 3.49-3.57 (m, 1H)

¹³C-NMR: 108.15, 105.93, 77.03, 75.38, 72.88, 72.85, 72.51, 71.08, 70.95, 70.91, 70.66, 68.57, 61.07, 59.46

¹⁹F-NMR: 150.64

### Example 3: General Fermentation Procedure

The culture was carried out in a 2 l fermenter containing 1 liter of minimal medium containing ammonium phosphate 87 mM, potassium phosphate 51 mM, TMS-N, Citric Acid 5.2 mM, potassium hydroxide 45 mM, sodium hydroxide 25 mM, magnesium sulphate 2.5 mM as well as Glucose 15.9 g/L and Glycerol 2.4 g/L as initial carbon source. The growth phase started with the inoculation (2% inoculum). The temperature was kept at around 33° C. and the pH regulated at 6.8 with aqueous NH₄OH solution. The oxygen was kept at 40% with an air flow between 0.5 to 3 L/min until cells were adapted to the glycerol in the medium. When all initial carbon source was depleted, the fed-batch phase was initiated, the exogenous precursor lactosyl fluoride (1b, 25 g/L) and the inducer IPTG (1-2 ml of a 50 ng/ml solution) were added to the culture and the temperature was decreased to 28° C. The fed-batch was realized using a 750 g/L aqueous glycerol solution, with a high substrate feeding rate of ≈4.5 g/h of glycerol for 1 l of culture. The culture was monitored by HPLC (see FIGS. 1A and 1B), and the identity of the peaks was confirmed by MS analysis. The maximal production yield for compounds 2d and 2e was observed after 48 h of fermentation.

### Example 4: General Purification Procedure

The fermentation broth was ultrafiltered (5-30 kDa membrane) at 25° C. until the total volume was concentrated to half and the UF permeate was collected. The UF retentate was then washed with purified water (4 to 5-fold volumes relative to the ultrafiltered broth volume) until all compound of interest was extracted to the permeate. The combined UF permeates were then subjected to nanofiltration (300-500 Da membrane) at 30 bar and 15° C. until the retentate reached a concentration 20 to 30-fold higher than the initial solution.

The NF retentate was subjected to standard chromatographic techniques to afford the final compounds.

### Example 5: Synthesis of α-N-acetylneuroaminoyl-(2→3)-β-D-galactopyranosyl-(1→4)-α-D-glucopyranosyl fluoride (2d)

Glycosyl fluoride 2d was obtained following the general fermentation and purification procedures described in examples 3 and 4. MS: 658.35 Da [M+Na]⁺;

¹H NMR (400 MHz, D₂O,) δ=5.69 (dd, J_{H,F}=53.4 Hz, J_{H,H}=2.7 Hz, 1H), 4.54 (d, J=7.7 Hz, 1H), 4.12 (dd, J=9.7, 3.2 Hz, 1H), 3.99 (m, 1H), 3.96 (m, 1H), 3.92 (m, 2H), 3.89 (m, 1H), 3.87 (m, 2H), 3.85 (m, 1H), 3.75 (m, 2H), 3.71 (m, 1H), 3.69 (m, 1H), 3.63 (m, 1H), 3.64 (m, 1H), 3.59 (m, 1H), 2.75 (dd, J=12.5, 4.7 Hz, 1H), 2.03 (s, 3H), 1.80 (t, J=12.5).

¹³C NMR (101 MHz, D₂O) δ=177.6, 176.5, 109 (d, J_{C-F}=222.0 Hz), 105.2, 102.4, 79.5, 78.1, 77.8, 75.5, 74.4, 73.7, 73.4 (d, J_{C-F}=25.2), 70.9, 70.1, 65.2, 63.7, 62.0, 42.2, 24.6.

¹⁹F NMR (376.5 MHz, D₂O): d=−150.6 (s) ppm

### Example 6: Synthesis of α-N-acetylneuroaminyl-(2→8)-α-N-acetylneuroaminyl-(2→3)-β-D-galactopyranosyl-(1→4)-α-D-glucopyranosyl fluoride (2e)

Glycosyl fluoride 2e was obtained following the general fermentation and purification procedures described in examples 3 and 4. MS: 949.45 Da [M+Na]⁺;

¹H NMR (400 MHz, D₂O) δ=5.57 (dd, J=53.4, 2.8 Hz, 1H), 4.42 (d, J=7.9 Hz, 1H), 4.12-3.93 (m, 3H), 3.90-3.40 (m, 23H), 2.66 (dd, J=12.4, 4.6 Hz, 1H), 2.56 (dd, J=12.3, 4.4 Hz, 1H), 1.94 (s, 4H), 1.91 (s, 4H), 1.62 (t, J=12.1 Hz, 2H) ppm.

¹³C NMR (101 MHz, D₂O) δ=174.95, 174.94, 173.47, 173.30, 107.00 (d, J=223.4 Hz), 102.59, 100.49, 100.13, 78.17, 76.64, 75.40, 75.21, 73.97, 72.87, 72.60, 71.71, 70.96, 70.89, 70.65, 69.25, 68.44, 68.07, 67.87, 67.42, 62.51, 61.51, 61.09, 59.31, 52.22, 51.70, 40.46, 39.67, 22.27, 21.99 ppm.

¹⁹F NMR (376 MHz, D₂O) δ=−150.63 (dd, J=53.4, 26.3 Hz) ppm.

### Example 7: Synthesis of α-Fucopyranosyl-(1→2)-β-D-galactopyranosyl-(1→4)-α-D-glucopyranosyl fluoride (2h)

Glycosyl fluoride 2h was obtained following the general fermentation and purification procedures described in examples 3 and 4. MS: 513.1 Da [M+Na]⁺.

### Example 8: Synthesis of β-N-Acetylamino-2-deoxy-β-glucopyranosyl-(1→3)-β-D-galactopyranosyl-(1→4)-α-D-glucopyranosyl fluoride (2i)

Glycosyl fluoride 2i was obtained following the general fermentation and purification procedures described in examples 3 and 4. MS: 570.2 Da [M+Na]⁺.

### Example 9: MS Analysis

The MS analysis was performed under the following conditions: ESI positive ionization, vaporizer temperature 300° C.; LC-MS mode, 1:1 split of flow; calibration with Pierce Triple Quadrupole Calibration Solution.

1. Method for producing a glycosyl fluoride of interest, the method comprising the steps of:
a) Providing an exogenous precursor and a genetically modified cell, wherein one or more glycosylation reactions can be performed on the exogenous precursor in the genetically modified cell, the genetically modified cell comprising one or more nucleic acid sequences encoding one or more glycosyltransferase enzymes, and wherein the exogenous precursor is a compound of General Formula I
X—F General Formula I,
wherein
X represents a glycosyl moiety,
F represents a fluorine atom
and
X and F are linked by an alpha or a beta glycosidic bond;
b) Culturing said genetically modified cell in a culture medium comprising said exogenous precursor, whereby
i. the exogenous precursor is internalized by the cell, and
ii. one or more glycosylation reactions are performed on the internalized exogenous precursor or on a glycosylated derivative thereof by the one or more glycosyltransferases, to form the glycosyl fluoride of interest,
c) Optionally isolating the glycosyl fluoride of interest from the genetically modified cell and/or from the culture medium.

2. The method according to claim 1, wherein the genetically modified cell is a yeast cell or a bacterial cell.

3. The method according to claim 1, wherein the one or more glycosyltransferase enzymes comprise one or more sialyltransferases and/or one or more fucosyltransferases, wherein the one or more glycosyltransferase enzymes are selected from the group consisting of β-1,3-N-acetylglucosaminyltransferase, β-1,6-N-acetylglucosaminyltransferase, β-1,3-galactosyltransferase, β-1,4-galactosyltransferase, β-1,4-N-acetylgalactosaminyltransferase, β-1,3-N-acetylgalactosaminyltransferase, β-1,3-glucoronosyltransferase, α-2,3-sialyltransferase, α-2,6-sialyltransferase, α-2,8-sialyltransferase, α-1,2-fucosyltransferase, α-1,3-fucosyltransferase, α-1,4-fucosyltransferase, α-1,4-galactosyltransferase, α-1,3-galactosyltransferase or a combination thereof.

4. (canceled)

5. The method according to claim 1, wherein the exogenous precursor is a compound of General Formula Ia: 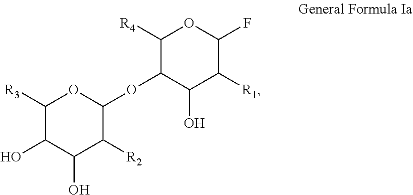
wherein
R₁ and R₂ are independently selected from the group consisting of OH, NH₂ and NH-acyl,
and
R₃ and R₄ are independently selected from the group consisting of —CH₂—OH and C₁₋₆ alkyl.

6. The method according to claim 1, wherein the exogenous precursor is a compound of General Formula Ib: 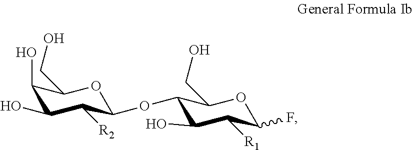
wherein R₁ and R₂ are independently selected from the group consisting of OH, NH₂ and NH-acyl.

7. The method according to claim 1, wherein the exogenous precursor is compound 1a: 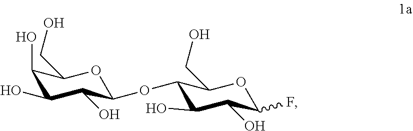
wherein glycosidic bond [symbol] is a glycosidic bond.

8. The method according to claim 1, wherein the genetically modified cell has no β-galactosidase activity.

9. The method according to claim 1, wherein X of General Formula I is a monosaccharide moiety, a disaccharide moiety or a trisaccharide moiety.

10. The method according to claim 1, wherein the glycosyl fluoride of interest is a compound selected from the group consisting of compounds defined by
(i) General Formula IIa: 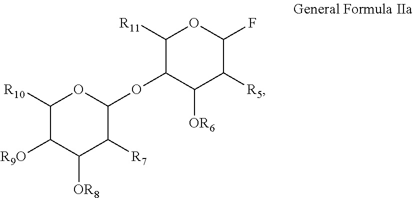
wherein
R₅ and R₇ are independently selected from the group consisting of OH, NH₂, NH-acyl and O-glycoside;
R₆, R₈ and R₉ are independently hydrogen or a glycosyl moiety;
and
R₁₀ and R₁₁ are independently selected from the group consisting of CH₂—OH, CH₂O-glycoside and C₁₋₆ alkyl,
(ii) General Formula IIb: 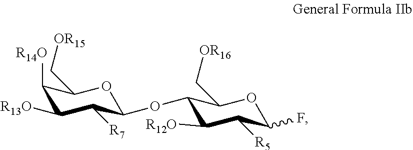
wherein
R₅ and R₇ are independently selected from the group consisting of OH, NH₂, NH-acyl and O-glycoside;
R₁₂, R₁₃, R₁₄, R₁₅ and R₁₆ are independently hydrogen or a glycosyl moiety,
(iii) General Formula Tic: 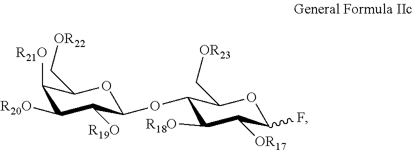
wherein
R₁₇, R₁₈, R₁₉, R₂₀, R₂₁, R₂₂, R₂₃ are independently hydrogen or a glycosyl moiety.

11. (canceled)

12. (canceled)

13. The method according to claim 7, wherein the glycosyltransferase enzyme is a α-2,3-sialyltransferase and the produced glycosyl fluoride of interest is compound 2a or a salt thereof: 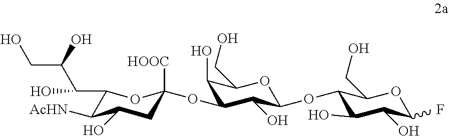
wherein glycosidic bond [symbol] is a glycosidic bond.

14. The method according to claim 7, wherein the glycosyltransferase enzymes are α-2,8-sialyltransferase and α-2,3-sialyltransferase, and the produced glycosyl fluoride of interest is compound 2b or a salt thereof: 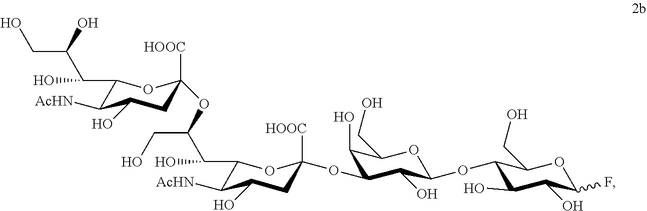
wherein glycosidic bond [symbol] is a glycosidic bond.

15. The method according to claim 6, wherein the glycosyltransferase enzymes are β-1,4-N-acetylgalactosaminyltransferase, β-1,3-galactosyltransferase and α-2,3-sialyltransferase, and the produced glycosyl fluoride of interest is compound 2c or a salt thereof: 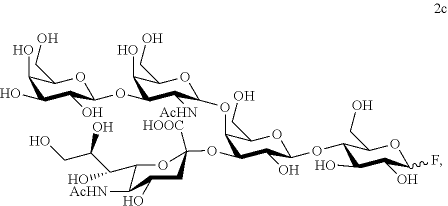
wherein glycosidic bond [symbol] is a glycosidic bond.

16. (canceled)

17. Compound of General Formula I:
X—F General Formula I,
wherein X represents a glycosyl moiety, F represents a fluorine atom,
X and F are linked by an alpha or a beta glycosidic bond,
and wherein the glycosyl moiety is selected from:
Neu5Acα2-3Galβ1-3GalNAcβ1-4Galβ1-4Glc1,
Neu5Acα2-3Galβ1,
Neu5Acα2-3Galβ1-3GalNAcβ1-4(Neu5Acα2-3)Galβ1-4Glc1,
Galβ1-3GalNAcβ1-4(Neu5Acα2-8Neu5Acα2-3)Galβ1-4Glc1,
Neu5Acα2-8Neu5Acα2-3Galβ1-3GalNAcβ1-4Gal 1-4Glc1,
Neu5Acα2-3Galβ1-3 (Neu5Acα2-6)GalNAcβ1-4Galβ1-4Glc1,
GalNAcβ1-4(Neu5Acα2-8Neu5Acα2-3)Galβ1-4Glc,
Neu5Acα2-8Neu5Acα2-3Galβ1-4Glc1,
Neu5Acα2-8Neu5Acα2-3Galβ1-3GalNAcβ1-4(Neu5Acα2-3)Galβ1-4Glc1,
Neu5Acα2-3Galβ1-3GalNAcβ1-4(Neu5Acα2-8Neu5Acα2-3)Galβ1-4Glc1,
Galβ1-3GalNAcβ1-4(Neu5Acα2-8Neu5Acα2-8 Neu5Acα2-3)Galβ1-4Glc1,
Neu5Acα2-3Galβ1-3(Neu5Acα2-6)GalNAcβ1-4(Neu5Acα2-3)Galβ1-4Glc1,
GalNAcβ1-4(Neu5Acα2-8Neu5Acα2-8 Neu5Acα2-3)Galβ1-4Glc1,
Neu5Acα2-8Neu5Acα2-8 Neu5Acα2-3Galβ1-4Glc1,
Neu5Acα2-8Neu5Acα2-3Galβ1-3GalNAcβ1-4(Neu5Acα2-8Neu5Acα2-3)Galβ1-4Glc1,
Neu5Acα2-3Galβ1-3(Neu5Acα2-6)GalNAcβ1-4(Neu5Acα2-8Neu5Acα2-3)Galβ1-4Glc1,
Neu5Acα2-3Galβ1-3GalNAcβ1-4(Neu5Acα2-8Neu5Acα2-8Neu5Acα2-3) Galβ1-4Glc1,
Galα1-3Galβ1-4Glc1-,
GalNAcβ1-3Galα1-4Galβ1-4Glc1-,
GalNAcβ1-3Galα1-3Galβ1-4Glc1-,
Galβ1-3GalNAcβ1-3Galα1-4Galβ1-4Glc1-,
Neu5Acα2-3Galβ1-3GalNAcβ1-3Galα1-4Galβ1-4Glc1-,
Neu5 Acα2-3Galβ1-3 (Neu5 Acα2-6)GalNAcβ1-3Galα1-4Galβ1-4Glc1-,
GalNAcα1-3GalNAcβ1-3Galα1-4Galβ1-4Glc1-,
GalNAcβ1-3GalNAcβ1-3Galα1-4Galβ1-4Glc1-,
GalNAcα1-3GalNAcβ1-3 (Galβ1-3GalNAcβ1-4)Gal a 1-4Galβ1-4Glc1-,
Galβ1-3GalNAcβ1-3Galα1-4Galβ1-4Glc1-,
Fucα1-2Galβ1-3GalNAcβ1-3Galα1-4Galβ1-4Glc1-,
GalNAcα1-3(Fucα1-2)Galβ1-3GalNAcβ1-3Galα1-4Galβ1-4Glc1-,
Galα1-3(Fucα1-2)Galβ1-3GalNAcβ1-3Galα1-4Galβ1-4Glc1-,
GlcNAcβ1-3Galβ1-4GlcNAcβ1-3Galβ1-4Glc1-,
Galβ1-4GlcNAcβ1-3Galβ1-4GlcNAcβ1-3Galβ1-4Glc1-,
Galβ1-4(Fucα1-3)GlcNAcβ1-3Galβ1-4Glc1-,
Galβ1-4(Fucα1-3)GlcNAcβ1-3Galβ1-4GlcNAcβ1-3Galβ1-4Glc1-,
Galβ1-4(Fucα1-3)GlcNAcβ1-3Galβ1-4(Fucα1-3)GlcNAcβ1-3Galβ1-4Glc1-,
Galβ1-4GlcNAcβ1-3(Galβ1-4GlcNAcβ1-6)Galβ1-4GlcNAcβ1-3Galβ1-4Glc1-,
Fucα1-2Galβ1-4GlcNAcβ1-3Galβ1-4Glc1-,
GalNAcβ1-3(Fucα1-2)Galβ1-4GlcNAcβ1-3Galβ1-4Glc1-,
GalNAcα1-3(Fucα1-2)Galβ1-4GlcNAcβ1-3Galβ1-4Glc1-,
Galα1-3(Fucα1-2)Galβ1-4GlcNAcβ1-3Galβ1-4Glc1-,
Fucα1-2Galβ1-3GalNAcα1-3(Fucα1-2)Galβ1-4GlcNAcβ1-3Galβ1-4Glc1-,
Fucα1-2Galβ1-4(Fucα1-3)GlcNAcβ1-3Galβ1-4Glc1-,
Fucα1-2Galβ1-4(Fucα1-3)GlcNAcβ1-3Galβ1-4GlcNAcβ1-3Galβ1-4Glc1-,
Galβ1-3(Fucα1-4)GlcNAcβ1-3Galβ1-4(Fucα1-3)GlcNAcβ1-3Galβ1-4Glc1-,
GalNAcβ1-3Galβ1-4GlcNAcβ1-3Galβ1-4Glc1-,
Galα1-3Galβ1-4GlcNAcβ1-3 (GalNAcβ1-4)Galβ1-4Glc1-,
GlcNAcβ1-3Galβ1-4(Fucα1-3)GlcNAcβ1-3Galβ1-4Glc1-,
Galα1-3Galβ1-4(Fucα1-3)GlcNAcβ1-3Galβ1-4Glc1-,
Galβ1-4Galβ1-4GlcNAcβ1-3Galβ1-4Glc1-,
GalNAcβ1-3Galα1-3Galβ1-4GlcNAcβ1-3Galβ1-4Glcβ1-,
Galβ1-3 GlcNAcβ1-3Galβ1-4Glc1-,
GlcNAcβ1-3Galβ1-4Glc1-,
Galβ1-3 GlcNAcβ1-3 (Galβ1-4GlcNAcβ1-6)Galβ1-4Glc1-,
Galβ1-4GlcNAcβ1-3 (Galβ1-4GlucNAcβ1-6)Galβ1-4Glc1-,
Fucα1-2Galβ1-4Glc1-,
Galβ1-4(Fucα1-3)Glc1-,
Fucα1-2Galβ1-4(Fucα1-3)Glc1-,
Fucα1-2Galβ1-3GlcNAcβ1-3Galβ1-4Glc1-,
Galβ1-3(Fucα1-4)GlcNAcβ1-3Galβ1-4Glc1-,
Galβ1-3(Fucα1-3)GlcNAcβ1-3Galβ1-4Glc1-,
Galβ1-3 GlcNAcβ1-3Galβ1-4(Fucα1-3)Glc1-,
Fucα1-2Galβ1-3(Fucα1-4)GlcNacβ1-3Galβ1-4Glc1-,
Neu5Acα2-6Galβ1-4Glc1-,
Neu5Acα2-3Galβ1-4(Fucα1,3)Glc1-,
Neu5Acα2-3Galβ1-3GlcNAcβ1-3Galβ1-4Glc1-,
Galβ1-3 (Neu5Acα2-6)GlcNAcβ1-3Galβ1-4Glc1-,
Neu5Acα2-6Galβ1-3GlcNAcβ1-3Galβ1-4Glc1-,
Neu5Acα2-3Galβ1-3 (Neu5Acα2-6)GlcNAcβ1-3Galβ1-4Glc1-,
Galβ1-3Galβ1-4Glc1-,
Galβ1-6Galβ1-4Glc1-,
Galβ1-3GalNAcβ1-4Galβ1-4Glc1-,
Fucα1-2Galβ1-3(Fucα1-4)GlcNAcβ1-3Galβ1-4Glc1-.

18. (canceled)

19. (canceled)

20. (canceled)

21. (canceled)

22. A compound selected from the group consisting of compounds defined by
(i) General Formula III: 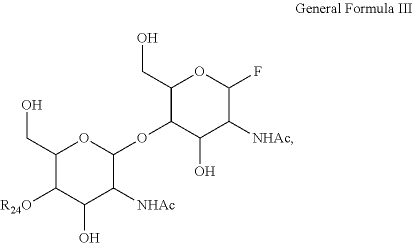
wherein R₂₄ is a glycosyl moiety;
(ii) General Formula IV: 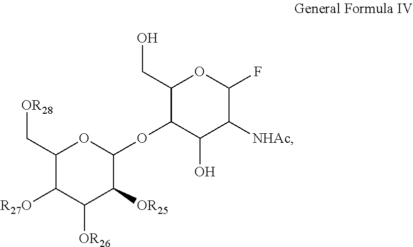
wherein
R₂₅, R₂₆, R₂₇ and R₂₈ are independently H or a glycosyl moiety.

23. (canceled)

24. (canceled)
